(19) United States
(12) Patent Application Publication
(10) Pub. No.: US 2025/0148292 A1
(43) Pub. Date: May 8, 2025

(54) ADVERSARIAL IMITATION LEARNING MODEL

(71) Applicant: NEC Laboratories America, Inc., Princeton, NJ (US)

(72) Inventors: LuAn Tang, Cranbury, NJ (US); Haoyu Wang, Plainsboro, NJ (US); Haifeng Chen, West Windsor, NJ (US); Wenchao Yu, Plainsboro, NJ (US); Zhengzhang Chen, Princeton Junction, NJ (US)

(21) Appl. No.: 18/620,125

(22) Filed: Mar. 28, 2024

Related U.S. Application Data
(60) Provisional application No. 63/596,683, filed on Nov. 7, 2023.
(60) Provisional application No. 63/627,200, filed on Jan. 31, 2024.

(51) Int. Cl. G06N 3/094; G06N 3/0455

(52) U.S. Cl. CPC G06N 3/094; G06N 3/0455

(57) ABSTRACT

Systems and methods train a transformer-based policy network and Generative Adversarial Network (GAN) by initializing a transformer-based policy network to model action sequences by encoding temporal dependencies within sensor data. Multi-head self-attention mechanisms process sequential sensor inputs by being pre-trained on a labeled dataset having sensor data from known low-risk action sequences. A generator within the GAN is trained to produce generated action sequences, which mimic behavior of low-risk action sequences. A discriminator within the GAN is concurrently trained to differentiate between action sequences derived from the labeled dataset and synthetic action sequences produced by the generator. A feedback loop is employed to adjust parameters to produce sequences indistinguishable from real low-risk action sequences. Risk scores are generated and low-risk action sequences are identified upon reaching a predetermined threshold for accuracy in distinguishing between real and synthetic action sequences.

### RELATED APPLICATION INFORMATION

This application claims priority to U.S. Provisional Application Nos. 63/596,683 filed Nov. 7, 2023 and 63/627,200, filed Jan. 31, 2024, incorporated herein by reference in their entirety.

This application is related to U.S. patent applications (Attorney Docket Number 23157MX) filed concurrently herewith.

### BACKGROUND

### Technical Field

The present invention relates to systems and methods for enhancing operational efficiency and risk management using Artificial Intelligence (AI), and more particularly to integrated systems and methods for AI-based enhancing of operational efficiency and risk management in industrial and insurance sectors using Performance-based Adversarial Imitation Learning (PAIL) for dynamically optimizing industrial processes and risk assessment in the insurance sector.

### Description of the Related Art

Monitored environments can be surveyed by large-scale sensors, and actions can be carried out by computer/artificial intelligence (AI). A problem arises in executing actions in an optimal sequence to achieve the best KPI (Key Performance Indicator) results. For example, in a steel making process, all the actions are controlled by computer, the KPI is carbon offset generated in the process. The AI generates an optimal action sequence to minimize the carbon offset by mining historical data. The input of the optimization system can include historical datasets of the production process. Each process sample can include the sensors that measure the environment, in a time series format, the sequences of actions conducted during the process and the final KPI result. The output is a trained model to monitor the sensor data and recommend actions based on recent sensor data, so that the process can achieve the optimized KPI (e.g., minimize the carbon offset generation).

Similar issues can arise when monitoring a large number of users or participants in a system. For example, monitoring driving records within a large population. A monitoring system is needed to determine circumstances that achieve a best KPI in the final result to make a determination or prediction. The search space may be very large (e.g., lots of drivers, homeowners, etc.). Further, for most applications, the KPI is numerical, and the actions are embedded with several numerical parameters. For example, the values of the parameters are directly related to the final KPI. Unfortunately, for most actions, the end users cannot provide a detailed evaluation on the effects or influences on the final KPI. The reward of each action is not clear. The system has to learn them from historical data.

### SUMMARY

According to an aspect of the present invention, a computer-implemented method for training a transformer-based policy network and Generative Adversarial Network (GAN) includes initializing a transformer-based policy network to model action sequences by encoding temporal dependencies within sensor data, where the transformer-based policy network utilizes multi-head self-attention mechanisms to process sequential sensor inputs, the multi-head self-attention mechanisms being pre-trained on a labeled dataset having sensor data from known low-risk action sequences; training a generator within the GAN to produce generated action sequences by feeding noise and latent representations of sensor data, where the generator employs an adapted transformer architecture to enhance diversity and realism of the generated action sequences, which mimic behavior of low-risk action sequences; concurrently training a discriminator within the GAN to differentiate between action sequences derived from the labeled dataset and synthetic action sequences produced by the generator; employing a feedback loop where assessments by the discriminator are used to adjust parameters for an iterative improvement process configured to produce sequences indistinguishable from real low-risk action sequences; and deploying the trained transformer-based policy network and GAN to process incoming unlabeled sensor data for real-time generation of risk scores and identification of low-risk action sequences upon reaching a predetermined threshold for accuracy in distinguishing between real and synthetic action sequences.

In other embodiments, training can include monitoring vehicles to collect the sensor data where the action sequences include driver actions. The action sequences can include historical driver actions. The sensor data can be cleaned and filtered by computing statistical measures on the sensor data to remove unrelated data by employing a Pearson correlation coefficient computation. The sensor data can include action sequences, and temporal correlations can be discerned using an adapted Transformer architecture to capture subtle and long-range dependencies within the action sequences. The discriminator can utilize a convolutional neural network (CNN) architecture optimized to identify subtle distinctions between real and generated action sequences. A performance prediction neural network can be used to assign risk scores to individual actions and identify low-risk action sequences, where the neural network is based on a Transformer architecture that performs analysis of actions in a context of their temporal sequence. An insurance premium for drivers can be estimated in accordance with the risk scores. The risk scores can be employed to directly adjust insurance policy terms for potential low-risk drivers.

According to another aspect of the present invention, a system for training a transformer-based policy network and Generative Adversarial Network (GAN) includes a hardware processor and a memory that stores a computer program. When executed by the hardware processor the program causes the hardware processor to initialize a transformer-based policy network to model action sequences by encoding temporal dependencies within sensor data, where the transformer-based policy network utilizes multi-head self-attention mechanisms to process sequential sensor inputs, the multi-head self-attention mechanisms being pre-trained on a labeled dataset having sensor data from known low-risk action sequences; train a generator within the GAN to produce generated action sequences by feeding noise and latent representations of sensor data, where the generator employs an adapted transformer architecture to enhance diversity and realism of the generated action sequences, which mimic behavior of low-risk action sequences; concurrently train a discriminator within the GAN to differentiate between action sequences derived from the labeled dataset and synthetic action sequences produced by the generator; utilize a feedback loop where assessments by the discriminator are used to adjust parameters for an iterative improvement process configured to produce sequences indistinguishable from real low-risk action sequences; and deploy the trained transformer-based policy network and GAN to process incoming unlabeled sensor data for real-time generation of risk scores and identification of low-risk action sequences upon reaching a predetermined threshold for accuracy in distinguishing between real and synthetic action sequences.

In other embodiments, the hardware processor can monitor vehicles to collect the sensor data where the action sequences include driver actions. The action sequences can include historical driver actions. The computer program can cause the hardware processor to clean the sensor data by computing statistical measures on the sensor data, and filter the sensor data to remove unrelated data by employing a Pearson correlation coefficient computation. The sensor data can include action sequences and temporal correlations that are discerned using an adapted Transformer architecture to capture subtle and long-range dependencies within the action sequences. The discriminator can utilize a convolutional neural network (CNN) architecture optimized to identify subtle distinctions between real and generated action sequences. The computer program can cause the hardware processor to utilize a performance prediction neural network to assign risk scores to individual actions and identify low-risk action sequences, where the neural network is based on a Transformer architecture that performs analysis of actions in a context of their temporal sequence. The system can estimate an insurance premium for drivers in accordance with the risk scores. The risk scores can be employed to directly adjust insurance policy terms for potential low-risk drivers. The transformer-based policy network can employ a self-attention mechanism that enables simultaneous processing of multiple trajectories for modelling complex driver behaviors over time.

According to another aspect of the present invention, a computer-readable medium storing instructions that, when executed by a processor, perform a method for training a transformer-based policy network and Generative Adversarial Network (GAN) that includes initializing a transformer-based policy network to model action sequences by encoding temporal dependencies within sensor data, where the transformer-based policy network utilizes multi-head self-attention mechanisms to process sequential sensor inputs, the multi-head self-attention mechanisms being pre-trained on a labeled dataset having sensor data from known low-risk action sequences; training a generator within the GAN to produce generated action sequences by feeding noise and latent representations of sensor data, where the generator employs an adapted transformer architecture to enhance diversity and realism of the generated action sequences, which mimic behavior of low-risk action sequences; concurrently training a discriminator within the GAN to differentiate between action sequences derived from the labeled dataset and synthetic action sequences produced by the generator; employing a feedback loop where assessments by the discriminator are used to adjust parameters for an iterative improvement process configured to produce sequences indistinguishable from real low-risk action sequences; and deploying the trained transformer-based policy network and GAN to process incoming unlabeled sensor data for real-time generation of risk scores and identification of low-risk action sequences upon reaching a predetermined threshold for accuracy in distinguishing between real and synthetic action sequences.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustratively depicting an exemplary processing system to which the present invention may be applied, in accordance with embodiments of the present invention;

FIG. 2 is a diagram illustratively depicting a high-level view of a system and method for offline training using Performance-based Adversarial Imitation Learning (PAIL) for dynamic optimization of industrial processes and personalized risk assessment, in accordance with embodiments of the present invention;

FIG. 3 is a diagram illustratively depicting a high-level view of a system and method for real-time online testing and monitoring using Performance-based Adversarial Imitation Learning (PAIL) for dynamic optimization of industrial processes and personalized risk assessment, in accordance with embodiments of the present invention;

FIG. 4 is a diagram illustratively depicting a system and method for optimizing Key Performance Indicators (KPI) for one or more systems and processes by integrating offline training and online testing devices using Performance-based Adversarial Imitation Learning (PAIL) for dynamic optimization of industrial processes and personalized risk assessment, in accordance with embodiments of the present invention;

FIG. 5 is a diagram illustratively depicting a system and method for dynamically optimizing industrial processes and personalized insurance risk assessment using Performance-based Adversarial Imitation Learning (PAIL), including discriminator and performance estimator networks for learning optimal action sequences and improving KPI prediction accuracy, in accordance with embodiments of the present invention;

FIG. 6 is a block/flow diagram illustratively depicting a method for dynamically optimizing industrial processes and personalized insurance risk assessment using Performance-based Adversarial Imitation Learning (PAIL), including discriminator and performance estimator networks for learning optimal action sequences and improving KPI prediction accuracy, in accordance with embodiments of the present invention;

FIG. 7 is a diagram illustratively depicting a framework of a system and method for action sequence generation using an Adversarial Imitation Learning Engine (AILE) for dynamic personalized driver action risk assessment, in accordance with embodiments of the present invention;

FIG. 8 is a diagram illustratively depicting a system and method for model training using an Adversarial Imitation Learning Engine (AILE) for dynamic personalized driver action risk assessment, in accordance with embodiments of the present invention;

FIG. 9 is a diagram illustratively depicting a high-level view of a system and method for real-time end user/customer searching and monitoring using an Adversarial Imitation Learning Engine (AILE) for dynamic personalized driver action risk assessment, in accordance with embodiments of the present invention;

FIG. 10 is a diagram illustratively depicting a framework of a system and method for dynamically optimizing personalized driver action risk assessment based on a distinguisher and risk estimator using an Adversarial Imitation Learning Engine (AILE), in accordance with embodiments of the present invention;

FIG. 11 is a diagram illustratively depicting a system and method for optimizing Key Performance Indicators (KPI) for one or more systems and processes by integrating offline training and online testing devices using an Adversarial Imitation Learning Engine (AILE) for dynamic personalized driver action risk assessment, in accordance with embodiments of the present invention;

FIG. 12 is a block/flow diagram illustratively depicting a method for optimizing Key Performance Indicators (KPI) for one or more systems and processes by integrating offline training and online testing devices using an Adversarial Imitation Learning Engine (AILE) for dynamic personalized driver action risk assessment, in accordance with embodiments of the present invention; and

FIG. 13 is a block/flow diagram illustratively depicting a method for training Artificial Intelligence (AI) models for dynamic, real-time personalized driver action risk assessment using an Adversarial Imitation Learning Engine (AILE), in accordance with embodiments of the present invention.

### DETAILED DESCRIPTION

In accordance with embodiments of the present invention, systems and methods are provided for dynamically optimizing industrial processes and personalized insurance risk assessment using Performance-based Adversarial Imitation Learning (PAIL), including discriminator and performance estimator networks for learning optimal action sequences and improving KPI prediction accuracy. In accordance with embodiments of the present invention, systems and methods provide a Performance based Adversarial Imitation Learning (PAIL) engine for KPI optimization, and apply it for evaluating risk.

In one example, systems and methods in accordance with the present embodiments can be employed to evaluate possibilities of risky action sequences without pre-defined knowledge on action rewards. In particularly useful embodiments, an action sequence such as driving behavior can be evaluated for individual drivers. The systems and methods in accordance with the present embodiments can employ a transformer-based policy generator network to forecast subsequent actions of drivers, homeowners, etc. based on historical data, and iteratively generate a prediction of future action sequences.

For numerical KPI optimization, embodiments put the processes into different classes of, e.g., high performance, low performance, etc. and learn a corresponding discriminator (e.g., in the format of a neural network) to select high performers (e.g., good drivers, low-risk homeowners, etc.). In one embodiment, the discriminator can be trained using high performers only. The systems and methods in accordance with the present embodiments can generate a distribution of the parameters and output the parameter values based on the distribution. The systems and methods in accordance with the present embodiments can learn another transformer-based performance prediction network to estimate the final KPI results. In this way, the reward of each action is computed as the difference of KPI.

In one embodiment, the systems and methods for risk assessment in accordance with the present embodiments can be employed to generate leads for insurance carriers. The leads can be identified by, e.g., historical data on individuals or companies with good driving records, low-risk property owners, etc. Once identified the insurance companies can reach out to the individuals with low-risk ratings to offer a discount or other incentives for signing up for an insurance policy through that carrier. Leads could be generated automatically and emails could be automatically generated for the purpose of offering discounts or reduced premiums to individuals with low risk assessments in order to attract this class of customers to increase the insurance base with low-risk individuals. The risk assessment can be employed in auto insurance but can be adapted for flood insurance, homeowners and other insurance types. Data can be collected for vehicles and stored in the cloud. Commercial vehicle data is a likely source of data and includes less privacy concerns.

To address the above challenges, embodiments of the present invention introduce a PAIL framework that employs a Transformer based autoencoder to learn policy and predict subsequent actions based on preceding operations. The systems and methods concurrently simulate a future state using current states and actions. In this way, an entire action sequence is iteratively generated. A discriminator aimed at minimizing the discrepancy between generated action sequences and real sequences with high KPI, facilitates the policy in mimicking successful operation processes. Furthermore, to leverage limited training samples, performance under normal samples with the certain constraints is employed. To this end, the present embodiments use another Transformer-based performance prediction neural network to estimate the final performance range based on the system status at any given step, thereby ensuring each action contributes to an understanding of performance. Consequently, the reward signals from both the discriminator and performance predictor collectively refine generated policy.

The present invention further relates to systems and methods for industrial operation optimization using artificial intelligence, and more particularly to an integrated system and method for enhancing carbon neutrality in industrial processes through Performance-based Adversarial Imitation Learning (PAIL). This novel approach can utilize a transformer-based policy generator model to forecast and iteratively generate optimal action sequences (e.g., for minimizing carbon emissions, predicting insurance risks for particular individuals or entities, etc.), without the need for predefined action rewards.

The present invention uniquely addresses the challenges of action sequence generation, handling of numerical data, and uncertain rewards in a variety of fields. By leveraging a discriminator to minimize discrepancies between generated sequences and high-performance real-world sequences, alongside a Q-learning framework-based performance prediction model to estimate the value of each action, the system can substantially boost, for example, carbon neutrality of industrial operations. This dual reward mechanism ensures the generation of action sequences that contribute effectively to sustainable development goals, making the present invention a crucial advancement in the field of artificial intelligence industrial systems and risk assessment systems. In some embodiments, a Performance-based Adversarial Imitation Learning (PAIL) system can be utilized for generating actionable insights and optimal action sequences to minimize carbon emissions and assess risk for generating insurance leads. This approach leverages transformer-based models and a dual reward mechanism, making it an important advancement for achieving sustainable industrial efficiency and enhancing insurance risk management, in accordance with aspects of the present invention.

In various embodiments, the present invention utilizes a novel Performance based Adversarial Imitation Learning (PAIL) framework for industrial operation optimization. PAIL can employ a transformer based autoencoder to learn the policy and predict subsequent actions based on preceding operations. PAIL can simultaneously simulate the future state using the current states and actions. In this way, an entire action sequence is iteratively generated through. A corresponding discriminator, configured for minimizing the discrepancy between generated action sequences and real sequences with high KPI, can be utilized to facilitate the policy in mimicking successful operation processes. Furthermore, to leverage limited training samples, the present invention can improve performance under normal samples with the certain constraints. To this end, PAIL can utilize another transformer-based performance prediction neural network to estimate the final performance range based on the system status at any given step, thereby ensuring each action contributes to a performance improvement. Consequently, the reward signals from both the discriminator and performance predictor collectively refine generated policy, in accordance with aspects of the present invention.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid-state memory, magnetic tape, a removable computer diskette, a random-access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

Aspects of the present invention are described below with reference to flowchart illustrations and/or block diagrams of methods, systems, and computer program products according to embodiments of the present invention. It is noted that each block of the flowcharts and/or block diagrams, and combinations of blocks in the flowcharts and/or block diagrams, may be implemented by computer program instructions.

The flowchart and block diagrams in the figures illustrate the architecture, functionality, and operation of possible implementations of systems, methods, and computer program products according to various embodiments of the present invention. Each block in the flowchart or block diagrams may represent a module, segment, or portion of code, which comprises one or more executable instructions for implementing the specified logical function(s), and in some alternative implementations of the present invention, the functions noted in the block may occur out of the order noted in the figures. For example, two blocks shown in succession may, in fact, be executed substantially concurrently, may sometimes be executed in reverse order, or may be executed in any other order, depending on the functionality of a particular embodiment.

It is also noted that each block of the block diagrams and/or flowchart illustration, and combinations of blocks in the block diagrams and/or flowchart illustration, can be implemented by specific purpose hardware systems that perform the specific functions/acts, or combinations of special purpose hardware and computer instructions according to the present principles.

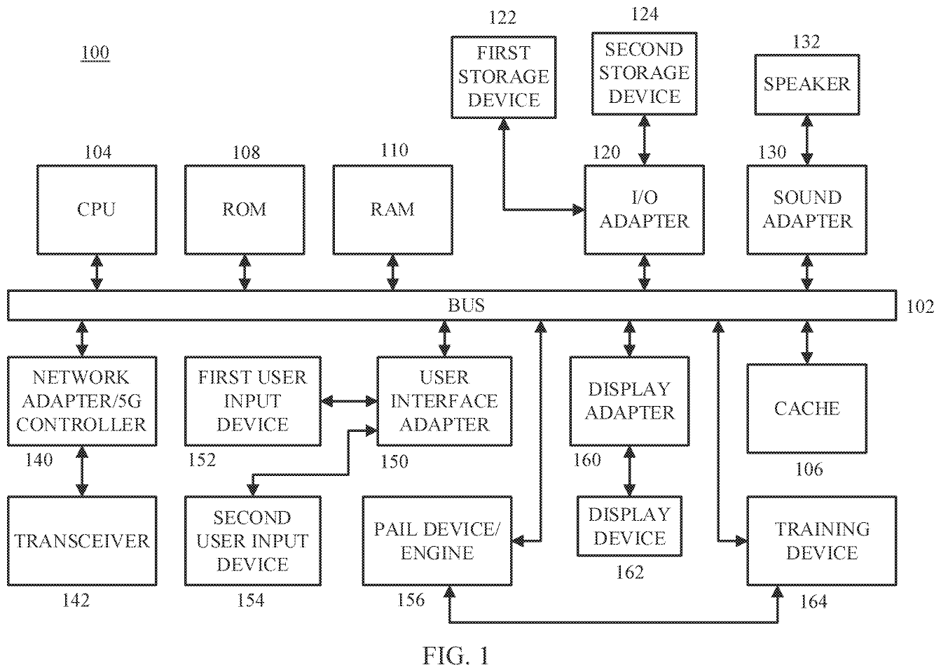

Referring now to the drawings in which like numerals represent the same or similar elements and initially to FIG. 1, an exemplary processing system 100, to which the present principles may be applied, is illustratively depicted in accordance with embodiments of the present principles.

In some embodiments, the processing system 100 can include at least one processor (CPU) 104 operatively coupled to other components via a system bus 102. A cache 106, a Read Only Memory (ROM) 108, a Random Access Memory (RAM) 110, an input/output (I/O) adapter 120, a sound adapter 130, a network adapter 140, a user interface adapter 150, and a display adapter 160, are operatively coupled to the system bus 102.

A first storage device 122 and a second storage device 124 are operatively coupled to system bus 102 by the I/O adapter 120. The storage devices 122 and 124 can be any of a disk storage device (e.g., a magnetic or optical disk storage device), a solid-state magnetic device, and so forth. The storage devices 122 and 124 can be the same type of storage device or different types of storage devices.

A speaker 132 is operatively coupled to system bus 102 by the sound adapter 130. A transceiver 142 is operatively coupled to system bus 102 by network adapter 140. A display device 162 is operatively coupled to system bus 102 by display adapter 160. A Performance based Adversarial Imitation Learning (PAIL) device 156 can be further coupled to system bus 102 by any appropriate connection system or method (e.g., Wi-Fi, wired, network adapter, etc.), in accordance with aspects of the present invention.

A first user input device 152 and a second user input device 154 are operatively coupled to system bus 102 by user interface adapter 150. The user input devices 152, 154 can be one or more of any of a keyboard, a mouse, a keypad, an image capture device, a motion sensing device, a microphone, a device incorporating the functionality of at least two of the preceding devices, and so forth. One or more video cameras can be included as input devices 152, 154, and the video cameras can include one or more storage devices, communication/networking devices (e.g., WiFi, 4G, 5G, Wired connectivity), hardware processors, etc., in accordance with aspects of the present invention. In various embodiments, other types of input devices can also be used, while maintaining the spirit of the present principles. The user input devices 152, 154 can be the same type of user input device or different types of user input devices. The user input devices 152, 154 are used to input and output information to and from system 100, in accordance with aspects of the present invention. A video compression device s user interface adapter 150 can process received video input, and a training device 164 (e.g., neural network trainer) can be operatively connected to the system 100 for controlling video codec for deep learning analytics using end-to-end learning, in accordance with aspects of the present invention.

Of course, the processing system 100 may also include other elements (not shown), as readily contemplated by one of skill in the art, as well as omitting certain elements. For example, various other input devices and/or output devices can be included in processing system 100, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 100 are readily contemplated by one of ordinary skill in the art given the teachings of the present principles provided herein.

Moreover, it is to be appreciated that the systems described below with respect to the FIGS., are systems for implementing respective embodiments of the present invention. Part or all of processing system 100 may be implemented in one or more of the elements of these systems, in accordance with aspects of the present invention.

Further, it is to be appreciated that processing system 100 may perform at least part of the methods described herein, in accordance with aspects of the present invention.

As employed herein, the term “hardware processor subsystem,” “processor,” or “hardware processor” can refer to a processor, memory, software, or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

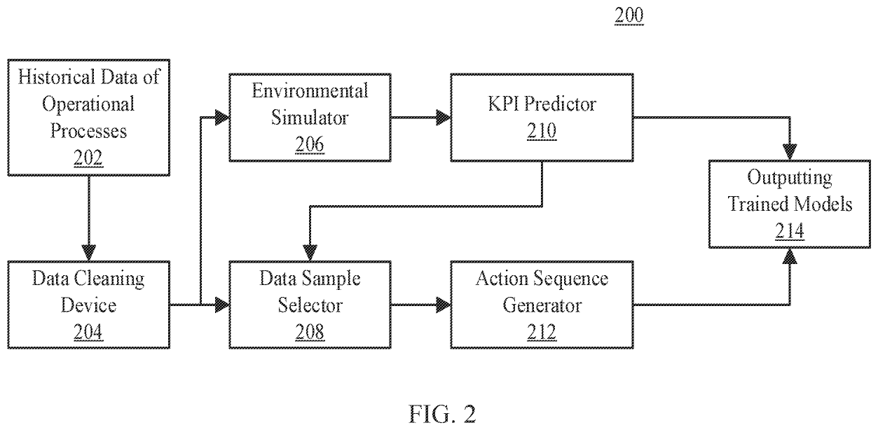

Referring now to FIG. 2, a high-level view of a system and method 200 for offline training using Performance-based Adversarial Imitation Learning (PAIL) for dynamic optimization of industrial processes and personalized risk assessment, is illustratively depicted in accordance with embodiments of the present invention.

In various embodiments, historical data of events and/or processes (e.g., industrial processes, driving events for particular users, environmental events, vehicle processes and events, etc.) can be input in block 202 for analysis and used in training. Received data can be acquired from sensors measuring systems, people, or environmental data (e.g., in time series format), and can include, for example, sequences of actions conducted during a particular process, which can be utilized for generation of a final Key Performance Indicator (KPI) generation result. The offline training method 200 can include taking historical data of operation processes and/or events as input, and can output trained models of KPI predictions and action sequence recommendations.

A data cleaning device can perform data cleaning of the data in block 204 by removing irrelevant data (e.g., determined irrelevant sensor data, events, etc.), and an environmental simulator 206 can be trained to simulate sensor data based on previous actions. A KPI predictor 210 can be trained to estimate a final KPI, and comparatively high (e.g., above a threshold level) KPI processes can be selected in block 208 for generating data samples. The samples with comparatively high KPI can be utilized to train an action sequence generator 212, and the KPI predictor 210 and the action sequence generator 212 can be output as trained models for subsequent use, in accordance with aspects of the present invention.

In various embodiments, in any of a plurality of types of physical systems (e.g., industrial operation system, vehicle navigation and control system, real-world event tracking system, etc.), a plurality of sensors can be utilized to capture any of a plurality of types of information related to the physical systems. Some sensors which can be utilized include, for example, condition sensors and controllable sensors.

Condition sensors can serve a read-only purpose, providing information about the environmental circumstances or the system situation and status. Controllable sensors, can be adjustable and can be modified in real time according to requirements of particular users or applications. The readings from the condition sensors can be regarded as the state, while adjustments made to the controllable sensors can be regarded as constituting an action. An objective of PAIL is to optimize the actions performed on systems to archive optimal KPI, in accordance with various aspects of the present invention. For convenience, particular notations utilized are provided below in Table 1:

TABLE 1

| Notation | Descriptions |
|---|---|
| [symbol] = {t} | The timestamp of actions |
| [symbol] = {sₜ} | The set of environment or system state |
| {aₜ} | The actions performed on system, [symbol] ∈ {R⁺}^{K} |
| | where k^{th} element denotes the k^{th} type of |
| | operation and its value indicates the value of |
| | corresponding operation. |
| [symbol] = {hₜ} | The historic state and action pair at step t, |
| | where hₜ = (s₁, a₁, . . . , Sₜ, aₜ) |
| T = {τ}ₙ | The set of trajectories consisting of the sequen- |
| | ces of states and actions where t = (s₁, a₁, . . . ) |
| Y = yₙ | The final KPI values for each trajectory |
| π_{E} (aₜ \| sₜ, hₜ₋₁) | The expert policy |
| π_{θ} (aₜ \| sₜ, hₜ₋₁) | The learnt policy parameterized by θ |
| ρ_{π}: [symbol] × [symbol] | The distribution of state-action pairs that the |
| | policy π interacts with the system |

In various embodiments, a primary objective of utilization of PAIL is to optimize the actions performed in an industrial system and/or assessing insurance risks for particular individuals or entities by refining learned models iteratively to reach an optimal KPI. During the model learning phase, an improvement of KPI can be estimated, taking the action as a reward signal. During the evaluation phase, KPI improvement can be utilized as a performance indicator. To facilitate this, the system can initially learn a function capable of predicting the KPI value. Consequently, the problem can be divided into two phases, as described in further detail herein below, in accordance with aspects of the present invention.

As an illustrative example, given a set of operation trajectories denoted by T={τ_1, τ_2, . . . , τ_n}, where each trajectory t consists of a history H and a corresponding y and the lengths of all trajectories are all N, the objective can be viewed as twofold. First, the final Key Performance Indicator (KPI) can be predicted given the complete sequence of actions and states, represented as ŷ=f (H). Second, an optimal policy π_{θ}(a, t|s, h) that can infer the current action and its timestamp based on the current state and historical trajectory can be learned and applied to archive optimal KPI for industrial operations and risk assessment for insurance operations. In practice, at a given timestamp t, the historical context ht−1 and the current state st, can be leveraged, and a subsequent action at can be generated with the learned policy π_{θ}(a, t|s, h). After obtaining the successor state from the probability distribution P(st+1|π, ht), an optimal sequence of actions can be iteratively generated for the upcoming time window.

Generative Adversarial Imitation Learning (GAIL) is a method that integrates aspects of both behavior cloning and inverse reinforcement learning using a generative adversarial network (GAN) based structure, which aims to minimize the Jensen-Shannon divergence between the expert policy RE and the generated policy TO as follows:

$${D}_{JS} ( {π}_{E} ‖ {π}_{θ} ) = \frac{1}{2} {D}_{KL} ( {π}_{E} ‖ \frac{{π}_{E} + {π}_{θ}}{2} ) + \frac{1}{2} {D}_{KL} ( {π}_{θ} ‖ \frac{{π}_{E} + {π}_{θ}}{2} ) \qquad ( 1 )$$

This model consists of two key components: a discriminator D and a policy generator π_{θ}. The generator aims to generate actions that mimic the expert's behavior, while the discriminator aims to distinguish between the agent's actions and the expert's actions.

Formally, the discriminator can solve the optimization problem as follows:

$${max}_{D ∈ {( 0 , 1 )}^{s \times 𝒜}} {𝔼}_{{π}_{E}} [ log D ( s , a ) ] + {𝔼}_{{π}_{θ}} [ log ( 1 - D ( s , a ) ) ] \qquad ( 2 )$$

where π_{E} is the expert's policy, π is the policy of the agent, and (s, a) are state-action pairs.

In contrast, the generator aims to ‘trick’ the discriminator by producing actions that are indistinguishable from those of the expert. This can be achieved by solving the following equation,

$${min}_{{π}_{θ}} {𝔼}_{π} [ log ( 1 - D ( s , a ) ) ] + λ H ( π ) \qquad ( 3 )$$

where H(π) is the entropy of the policy, and λ≥0 is a coefficient that encourages exploration by the agent. By alternating between training the discriminator and the generator, GAIL learns a policy n that is able to mimic the expert's behavior.

In some embodiments, a data cleaning device 204 (cleaning unit) can be used to pre-process the data and filter out the noisy condition sensors. In industrial, vehicle, and home systems, there can be a large number of sensors monitoring the environment and generating readings with not all of them are related to the final KPI. For each conditional sensor, the statistics of the time series, including the average, standard deviation, max value and min value, can be retrieved, an example being represented in Table 2 below. A Pearson correlation of each feature can be computed for the final KPI. If the absolute value of correlation is comparatively close to 0, it can indicate that the value of sensor is not related to final KPI, and PAIL will filter out such a sensor, in accordance with aspects of the present invention.

Table 2 lists the statistics of an exemplary environment, including sensor s1 to final KPI. In the historical dataset, there are 90 sample processes. For each process, PAIL retrieves the sensor data (time series) of s1 and computes the statistics (average, standard deviation (STD), maximum value (MAX), minimum value (MIN), median value (Median) as shown in Table 2, which illustrates statistics of sensor s1 with a final KPI. Then, PAIL can compute the person coefficient of each statistic to the final KPI. In this table, the coefficient between average value and final KPI is 0.76, and the coefficient between median and final KPI is 0.78. It indicates that the value of sensor s1 has a strong relationship with the final KPI. Hence, sensor s1 will not be filtered by the data cleaning module.

TABLE 2

| | Average | STD | Max | Min | Median | Final KPI |
|---|---|---|---|---|---|---|
| Sample 1 | 66 | 21 | 90 | 60 | 60 | 70 |
| Sample 2 | 64 | 12 | 80 | 55 | 56 | 68 |
| Sample 3 | 53 | 17 | 70 | 45 | 47 | 57 |
| . . . | . . . | . . . | . . . | . . . | . . . | . . . |
| Sample 90 | 80 | 16 | 96 | 67 | 75 | 75 |

An environmental simulator can be used in block 206 for the offline training 200 by simulating a particular environment. The decision-making process for industrial processes and insurance related risk assessment generally includes precise predictions regarding evolution of states following any given action. This forecasting aids in the generation of sequential industrial trajectories that can optimize or adhere to certain desirable outcomes.

In modeling this predictive framework, the present invention can utilize a Variational Autoencoder (VAE), which is a deep generative model that captures potential future states following an action. For a given state st and a designated action at, the encoder of the VAE can map the state-action pairing into a latent space as:

$${q}_{ϕ} ( z \vert {s}_{t} , {a}_{t} ) = 𝒩 ( z ; μ ( {s}_{t} , {a}_{t} ) , {σ}^{2} ( {s}_{t} , {a}_{t} ) )$$

where μ and σ are mean and variance of z. A core objective during training is to approximate the true posterior distribution. It seeks to minimize the discrepancy between predicted future states and observed outcomes, formulated as pθ(st+1|st, at, z).

The VAE can integrate a regularization term to prevent overfitting, thusly reducing processor requirements and increasing processing speed during use, and ensuring a smoother latent space by:

$$ℒ = {𝔼}_{{q}_{ϕ}} [ log {p}_{θ} ( {s}_{t + 1} \vert {s}_{t} , {a}_{t} , z ) ] - β KL ( {q}_{ϕ} ( z \vert {s}_{t} , {a}_{t} ) ‖ 𝒩 ( 0 , I ) )$$

where β is a hyper-parameter to balance the two terms in the loss function. The environmental simulator model 206 can be trained prior to the implementation of the imitation learning framework. Once it achieves a predefined performance criterion, its parameters can be fixed to ensure consistent interactions during subsequent learning stages. This simulation framework can be utilized to predict the consequences of certain actions, thereby informing the decision-making process and crafting optimal operational sequences, in accordance with aspects of the present invention.

In block 210, a KPI predictor/estimator can be utilized to construct a time series model configured for assimilating sequences from operational processes and driving events and processes, and forecasting their consequent outcomes. To this end, a refined self-attention mechanism can be utilized to discern the chronological interdependencies among states and actions across temporal intervals, thereby facilitating a comprehensive assessment of the operational process or driving event or processes' aggregate value. The network architecture can be similar to the structure of encoder of a policy generator, and for each trajectory t with length of N, the equation of calculating the whole sequence value (V) can be simplified as:

V(τ)=Self Attn(hN)

In numerous industrial and driving scenarios, quantifying the utility of discrete actions within a sequence is non-trivial, especially when only the cumulative outcome is discernible. This challenge of attributing credit to distinct actions can be accounted for by exploiting Temporal Difference (TD) learning for the development of a Q-value Network. This network can estimate the utility of a given state-action pairing, can include a reward (r) at timestep t, and is calculated as:

$${r}_{t} = - {E}_{( s , a ) ∼ {π}_{θ}} log ( D ( {s}_{t} , {a}_{t} ) )$$

The Q-network, initiated with arbitrary parameters, can be subsequently refined based on the Temporal Difference (TD) error, denoted as δt:

$$δ t = {r}_{t + 1} + γ Q ( {s}_{t + 1} , {a}_{t + 1} ) - Q ( {s}_{t} , {a}_{t} )$$

where γ embodies the discount factor, reflecting the contemporaneous valuation of prospective rewards, Q(st, at) signifies an approximation for the value of executing action at in state st, and Q(st+1, at +1) denotes the predicted value of the ensuing state-action pair.

A primary goal of TD learning is to minimize this TD error. Thus, the Q-value function can undergo iterative adjustments:

$$Q ( st , at ) ← Q ( st , at ) + αδ t$$

where α represents the learning rate. An additional objective is to curtail the expected squared TD error across trajectories propagated by policy πθ:

$$ℒ ( θ ) = {𝔼}_{( s , a ) ∼ {π}_{θ}} [ {δ}_{t}^{2} ] ,$$

and subsequent parameter refinement can be executed through gradient descent:

$$θ ← θ - η ∇ θ L ( θ )$$

with η indicating the gradient descent's step size.

The present invention can optimize a neural network architecture to infer the utility of diverse state-action pairings, and a guiding heuristic for this training emphasizes the maximization of the Q-value for actions chosen by the resultant policy:

$$Lvalue = - Ea ∼ πθ ( \cdot \vert s ) [ Q ( s , a ) ]$$

Therefore, the composite loss function for the policy generator becomes:

$$L = λ1 LIL + λ2 Lvalue$$

with λ1 and λ2 as hyper-parameters moderating the relative importance of the twin objectives. As a result, the policy generator can derive its learning signal through backpropagation from both the discriminator and the performance estimator. This dual influence can ensure that the generator not only emulates expert behavior but also ensures each action is optimized for value, in accordance with aspects of the present invention.

A data sample selector can be utilized in block 208 to select data for use in training. Not all the samples are used to train the model for action sequence generation, but rather only those with top KPI (e.g., above a particular threshold) are selected. The data sample selector is thus configured to automatically filter out the low KPI samples based on the threshold set (e.g., default or user-defined threshold).

In practice, it is noted that samples with high KPI are quite limited in real-world applications, and samples with middle and low KPI may still have some good segments to learn. For the samples with middle and low KPIs, the data sample selector can call the KPI estimator 210 to estimate the KPI improvements/changes at every time window. The segments or windows with high KPI improvements can also be selected and incorporated into the training sample in this manner. Next, the data sample selector 208 can output the filtered samples of comparatively high KPI for further processing using an action sequence generator 212.

In various embodiments, it is noted that in many industrial settings, vehicle operation settings (e.g., for assessing insurance risk levels), home device settings, etc., decision making processes are heavily contingent on historical states and actions. As an exemplary illustration, during oil extraction, a prior action, such as shutting off a gas valve, can significantly shape the probabilities of subsequent actions on the same equipment (e.g., shutting it off again or initiating a lift). In various embodiments, such temporal dependencies can be paramount, and can be accounted for by adaptation of a transformer architecture, which can be specifically configured and applied for discerning temporal correlations within time-series data.

In the domain of insurance risk assessment, temporal dependencies can play a role in understanding and predicting future events. As an example, consider an insurance company assessing the risk associated with insuring a fleet of vehicles. Similar to the above oil extraction example, historical states and actions can be pivotal in determining the likelihood of future events such as accidents or claims.

For example, if in the past there have been instances of reckless driving behavior within the fleet (or for an individual), such as speeding or frequent traffic violations, these historical actions can significantly influence the probability of future accidents and insurance claims. Moreover, the implementation of safety measures or driver training programs subsequent to such incidents can further alter the risk landscape. Additionally, environmental factors like weather conditions or the time of day can also influence the likelihood of accidents. For example, driving during adverse weather conditions like heavy rain or snow increases the risk of accidents compared to driving in clear weather, and such factors can be considered in assessing insurance risk levels for particular drivers or entities.

In this context, sophisticated risk assessment models of the present invention can account for these temporal dependencies and historical actions to accurately predict and mitigate potential risks. By leveraging advanced machine learning techniques, such as transformer architectures tailored for temporal data analysis, insurance companies can better understand the underlying patterns and correlations within time-series data, thus enhancing their ability to assess and manage insurance risks effectively, in accordance with aspects of the present invention.

In various embodiments, a core strength of the Transformer model lies in its multi-head self-attention mechanism. This feature provides the model with the capability to assign varying significance to different elements in a sequence relative to a focal point. Such a structure can be crucial to efficiently depicting intricate long-term dependencies inherent in time series datasets. Moreover, as an objective of the present invention revolves around the recurrent prediction of actions under the purview of the current state, the model's dexterity in handling sequences of fluctuating lengths can be beneficial in practice.

For example, for trajectory τl, let us define an input sequence of duration T as Xl={x1, x2, . . . , xT}. Here, each xt∈Xl represents the concatenated state and action vectors, st and at, at time t. The primary step involves the projection of the input xt into spaces of query Q, key K, and value V via unique projection matrices Wq, Wk, and Wv∈RT×d. The relevance of antecedent states is computed as,

$${Z}_{l} = {w}_{v} ( {X}_{l} {W}_{v} ) , {w}_{v}^{ij} = \frac{exp ( {e}_{v}^{ij} )}{{\sum }_{k = 1}^{T} exp ( {e}_{v}^{ik} )} ,$$

$${e}_{v}^{ij} = ( \frac{{( ( {X}_{l} {W}_{q} ) {( {X}_{l} {W}_{k} )}^{T} )}_{ij}}{\sqrt{{d}^{k}}} + {M}_{ij} )$$

where M∈RT×T is a mask matrix encoding temporal order,

This structure ensures that future events are not factored into the computation of prior state relevance. It is important to highlight that, given a goal to recurrently predict complete sequences, the length of the input sequence Xl is not fixed. To reconcile this, zeropadding can be employed to standardize the length of all inputs Xl to that of fully realized trajectories, denoted as N. The mask matrix ensures that these padded values remain inert during the attention computation. In the foundational Transformer model, positional information can be incorporated using sinusoidal position encodings. For position p in the sequence, the encoding can be computed for each dimension i as,

$${M}_{ij} = \{ \begin{matrix}0 , & if i \leq j \\ - ∞ , & if i > j\end{matrix}$$

$${PE}_{( pos , 2 i )} = sin ( \frac{pos}{{10000}^{2 i / d}} )$$

$${PE}_{( pos , 2 i + 1 )} = cos ( \frac{pos}{{10000}^{2 i / d}} )$$

where d is the dimensionality of the input embedding. The positional encoding matrix P is then element-wise added to the sequence Xl, furnishing the input representation H0 for the Transformer.

Within the Transformer paradigm, these representations can traverse L layers, subjected to both multi-head self-attention and position wise feed-forward operations: Formally, for each layer l=1, . . . , L

$${H}^{l} = LayerNorm ( {H}^{l - 1} + MultiHead ( {Q}^{l - 1} , {K}^{l - 1} , {V}^{l - 1} ) ) ,$$

$${H}^{l} = LayerNorm ( {H}^{l} + FFN ( {H}^{l} ) ) .$$

where LayerNorm is the layer normalization network and FFN is the position-wise feed-forward network. The multi-head attention mechanism segments the input H into h partitions, amalgamating outputs from these individual heads:

$$MultiHead ( Q , K , V ) = Concat ( head 1 , ... , headh ) Wo ,$$

where head i=Attention (Qi, Ki, Vi) and WO as another learned projection matrix.

In various embodiments, upon deploying the Transformer encoder on the temporal input data, the extracted representation HL encapsulates the intricate temporal interdependencies across various timestamps. Capitalizing on this, a subsequent dense neural network can be employed to predict the imminent action conditioned on the prevailing state. In this unique setup, the present invention is faced with a continuous action space encompassing multiple interrelated action types. Unlike traditional scenarios that employ the sigmoid function for categorical action selection, the present invention can assign a specific value to each type of action, to get the action vectors. These action sets adhere to a multivariate distribution, and consequently, current actions a from this distribution learnt by dense networks can be sampled as:

$${π}_{θ} ( a \vert h ) = Sample ( p ( a ; μ , \sum ) )$$

$$μ , \sum = Dense ( {H}^{L} )$$

where μ∈RK and Σ∈RK×K represent the mean vector and covariance matrix of the actions, respectively.

Adversarial Imitation Learning shares some common features with the foundational principles of Generative Adversarial Networks to train policies in reinforcement learning. Just as GANs use a discriminator to distinguish between real and generated samples, AIL uses a discriminator to distinguish between the real-world industry trajectories produced and those produced by the current policy. By trying to obfuscate this discriminator, the policy can adeptly learn to imitate the expert behavior. Here the present invention can leverage the @-parameterized multiple-layer perception (MLP) Dω(s,a), and this function estimates the likelihood that a given state action pair, (s,a), originates from genuine expert demonstrations.

Given that the discriminator is addressing a binary classification task, both the policy generator and the discriminator can engage in a min-max game, predicated on the cross-entropy loss as follows:

The objective of discriminator:

$${max}_{ω} {𝔼}_{{π}_{E}} [ log {D}_{ω} ( s , a ) ] + {𝔼}_{{π}_{θ}} [ log ( 1 - {D}_{ω} ( s , a ) ) ]$$

The objective of policy generator:

$${L}_{IL} = {min}_{θ} {𝔼}_{{π}_{θ}} [ log ( 1 - {D}_{ω} ( s , a ) ) ]$$

In the context of industrial scenarios, vehicle operation scenarios (e.g., for assessing insurance risk levels), home event scenarios, etc., optimal operational processes are scarce, posing significant challenges to imitation learning frameworks. Conventionally, even expert demonstrations do not epitomize the apex of operational efficiency, thereby not reaching the theoretically possible optimal performance. Noting this backdrop, the present invention can incorporate a performance oriented training guidance mechanism for the policy generator. A key of this mechanism is iteratively attempting to maximize the cumulative value of each state-action pair produced by the policy, thereby elevating the overall quality of generated trajectories.

The preliminary phase of this mechanism can necessitate the derivation of a specific reward signal for every discrete time step. Specifically, the output of discriminator can be interpreted as an inverse measure of the policy's performance. Formally, the reward signal derived from the discriminator for a given state-action pair (s,a) can be quantified as R(s,a)=−log D(s,a). This formulation shows a relationship in which when the discriminator assigns a value comparatively close to 0 to a particular state-action pair, suggesting that the pair diverges significantly from expert behavior, the corresponding reward is consequently a comparatively large negative value. This naturally penalizes the policy generator for actions that are perceived as non-expert, guiding the policy towards more expert-like decisions, in accordance with aspects of the present invention.

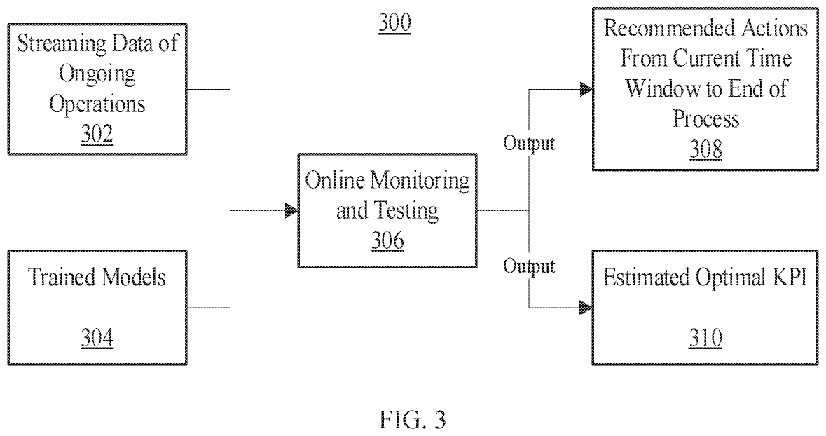

Referring now to FIG. 3, a diagram showing a high-level view of a system and method 300 for real-time online testing and monitoring using Performance-based Adversarial Imitation Learning (PAIL) for dynamic optimization of industrial processes and enhancing personalized risk assessment actions for the insurance industry, is illustratively depicted in accordance with embodiments of the present invention.

In various embodiments, the system architecture, designated broadly at numeral 300, embodies the real-time application of a Performance-based Adversarial Imitation Learning (PAIL) engine, in accordance with aspects of the present invention. Numeral 302 refers to the Streaming Data of Ongoing Operations, encapsulating real-time sensor readings that monitor various parameters pertinent to the operational processes. This continuous stream of data facilitates responsiveness and informed decision-making processes within the framework. Trained Models component 304 serves as a repository for the sophisticated algorithms developed during the offline training phase of the PAIL engine. These models incorporate the policy generator and the KPI predictor, among other elements, that have been refined using historical datasets of industry operations.

In various embodiments, the Online Monitoring and Testing module 306 represents where the real-time data and the pre-trained models converge. This module 306 assesses current conditions and predicts the immediate implications of potential actions. It takes the streaming data, juxtaposes it with the learned models, and computes the appropriate action sequences along with the prospective KPI outcomes. The output from the Online Monitoring and Testing module bifurcates into two distinct pathways. The first pathway leads to block 308, denoted as Recommended Actions From Current Time Window to End of Process. This channel can be pivotal for the generation of action sequences that are predicted to yield the most favorable KPI results, given the current operational state.

In various embodiments, the second pathway results in the Estimated Optimal KPI 310, and is the quantitative forecast of the KPI, predicated on the assumption that the recommended actions are implemented in practice. It is an evaluative output that measures the efficiency and effectiveness of the proposed action sequences within the given operational context. Collectively, the components of FIG. 3 embody the operational synthesis of the PAIL framework. The components of FIG. 3 demonstrate an orchestrated process wherein real-time operational data is continuously evaluated against a backdrop of sophisticated, pre-trained models. The output is a multi-faceted recommendation that not only proposes a sequence of actions aimed at achieving optimal operational performance but also quantifies the expected outcomes in the form of an estimated KPI and can execute corrective actions automatically (e.g., turn on/off components of an industrial system/vehicle, provide AI navigation and driving assistance to improve driving safety, etc.). This multi output mechanism ensures that the operational decisions are both proactive and grounded in robust analytical predictions, thus exemplifying a tangible advancement in the realm of industrial automation and risk management within the insurance industry. This architecture underpins the system's ability to adaptively optimize actions in pursuit of KPI maximization (e.g., carbon offset minimization), as well as enhancing risk assessment actions, all while addressing the challenges of large action sequence spaces, numerical data processing, and uncertain reward structures, in accordance with aspects of the present invention.

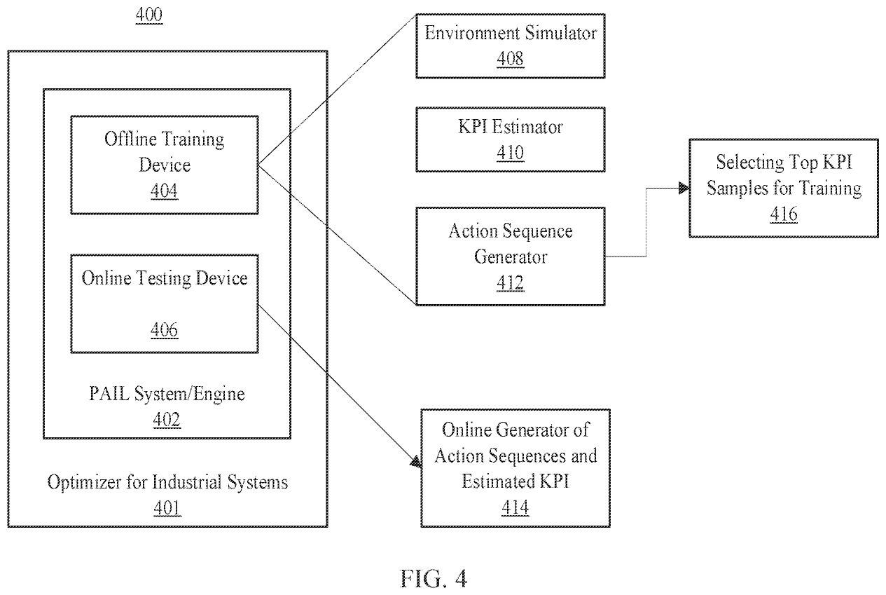

Referring now to FIG. 4, a diagram showing a system and method 400 for optimizing Key Performance Indicators (KPI) for one or more systems and processes by integrating offline training and online testing devices using Performance-based Adversarial Imitation Learning (PAIL) for dynamic optimization of industrial processes and personalized risk assessment, is illustratively depicted in accordance with embodiments of the present invention.

In various embodiments, as part of an optimizer for industrial systems 401, the PAIL engine 402 can include an offline training device 404 and an online testing device 406, each configured to enhance operational efficiency and KPI optimization through advanced AI methodologies, in accordance with aspects of the present invention. In various embodiments, the offline training device 404 integrates an environment simulator 408 designed to predict future states of industrial operations using processed historical sensor data. The environment simulator 408 employs a Variational Autoencoder (VAE) to project state-action pairs into a predictive latent space, facilitating the generation of accurate operational forecasts.

Further incorporated within the offline training device 404 can be a KPI Estimator 410 which evaluates and estimates the impact of action sequences on final KPI results. The KPI Estimator 410 can operate using a transformer-based model, deriving sophisticated reward computations for each action in relation to the KPI improvements, thereby supporting the identification and prioritization of actions that contribute to optimal process outcomes.

In some embodiments, also part of the offline training phase is an Action Sequence Generator 412, the forecasted future states are utilized from the environment simulator 408 to iteratively develop sequences of actions aimed at achieving the highest possible KPI performance. The Action Sequence Generator 412 harnesses the transformer-based architecture's dynamic input sequence adjustment capability to refine its predictive action sequencing. The training process can further involve a component for Selecting Top KPI Samples for Training 416, which filters historical process samples to identify and utilize only those with the most significant KPI outcomes. This selective approach ensures the training of the PAIL engine 402 on high-performance data, optimizing the learning process for quality rather than quantity of data.

In various embodiments, within the online testing device 406, the PAIL engine 402 transitions to applying the trained models in real-time. This can involve an Online Generator of Action Sequences and Estimated KPI 414 that processes streaming sensor data to recommend optimal actions and predict resultant KPIs. This online component enables dynamic, real-time decision-making that continuously adapts to live operational data for ongoing process optimization. The system 400 can store all historical datasets, processed sensor data, and developed training models within a memory component, ensuring the availability of robust data for both offline training and online action recommendation and associated performance of corrective actions, thereby underpinning the system's capacity for continuous learning and adaptation in varying industrial and insurance risk assessment and lead generation scenarios, in accordance with aspects of the present invention.

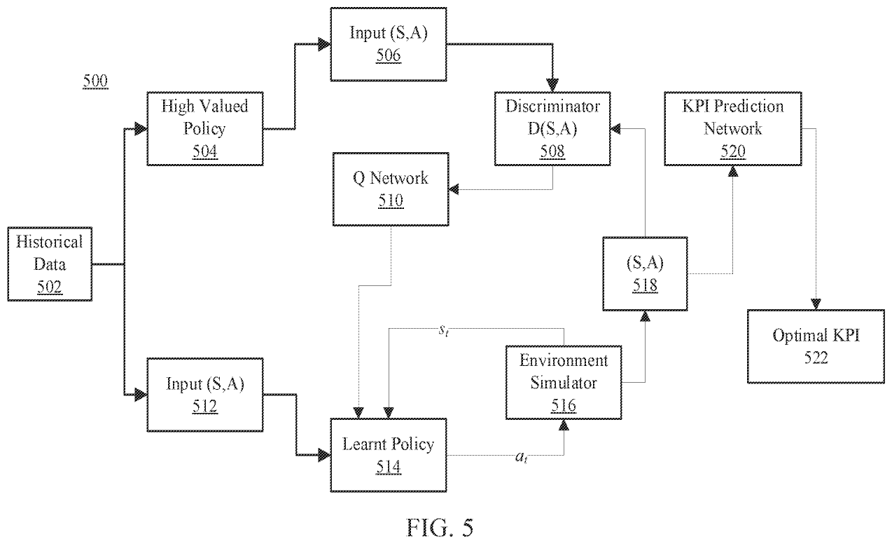

Referring now to FIG. 5, a diagram showing a system and method 500 for dynamically optimizing industrial processes and personalized insurance risk assessment using Performance-based Adversarial Imitation Learning (PAIL), including discriminator and performance estimator networks for learning optimal action sequences and improving KPI prediction accuracy, is illustratively depicted in accordance with embodiments of the present invention. FIG. 5 illustrates a multifaceted system for optimizing Key Performance Indicators (KPIs) within industrial settings to, e.g., achieve carbon neutrality or other applications, augment the efficacy of risk assessment actions (e.g., in the insurance industry). The depicted model synergizes various computational methodologies, including policy generation via deep learning, environmental state simulation, and reinforcement learning-based policy optimization, to inform the selection and generation of action sequences that propel the system towards the apex of operational efficiency and sustainability.

In various embodiments, the PAIL model can operate on input historical data, 502, which encapsulates previous state and action outcomes. This data repository is integral for establishing the baseline from which the model can extrapolate and learn. The historical data informs two concurrent input streams within the model: a high-valued policy input 506 and a learned policy input 512. Comparatively high-valued policies (e.g., above a threshold-user set or default), as referenced by numeral 504, represent the paradigmatic actions sequences previously determined to yield optimal outcomes.

The input state and action pairs (S, A) 506, are processed by a Discriminator module 508, and a Q Network 510. The Discriminator module 508 serves to evaluate the authenticity and effectiveness of the state-action pairings against the high-valued policy exemplars. In parallel, the Q Network 510 appraises the putative value of these pairings, providing a quantitative measure of their contribution towards the attainment of the system's KPIs. An Environment Simulator 516, utilizes the current state st and action at to forecast subsequent states. This simulator provides a dynamic replication of the system's response to actions, facilitating a forward-looking perspective for the anticipation of future system states and the subsequent optimization of actions. Complementarily, the KPI Prediction Network 520, integrates the evaluated state-action pairs (S,A) 518, to project future performance indicators. This predictive modeling gauges the long-term impact of operational decisions, providing a forward-projected KPI trajectory against which real-time decisions can be measured and refined, in accordance with aspects of the present invention.

The model converges representing optimized KPI 522, which synthesizes insights from the Discriminator module 508, the Q Network 510, and the KPI Prediction Network 520. This optimized KPI 522 embodies the model's predictive conclusions, representing the most favorable performance outcomes attainable from the current operational paradigm. It is the objective function of the PAIL model, providing a target for the system to strive towards through iterative learning and action refinement.

On the counterpart, input 512 marks the entry point for state and action pairs into the learned policy, designated learnt policy 514. This learnt policy 514 is iteratively refined through exposure to a variety of simulated and real-world inputs, allowing for the nuanced understanding and incorporation of complex operational dynamics within its decision-making processes. The learnt policy 514 is an outcome of continuous training and adaptation, influenced by the Discriminator's feedback loop and the Q Network's value estimations. As the learned policy evolves, it gravitates towards the high-valued policy ideal, with the end goal of autonomously generating state-action pairs that contribute to the attainment of the KPIs, with specific focus on, e.g., carbon neutrality for industrial applications and, e.g., the precision of risk assessments within the insurance industry.

In various embodiments, the iterative loop formed by numerals 514, 516, 518, 508, and 510 represents the continuous learning and adaptation cycle within the PAIL model. The system's capability to simulate environmental reactions to actions and predict the impact on KPIs allows for a robust policy generation mechanism that is sensitive to both immediate and long-term operational parameters. FIG. 5 encapsulates a complex adaptive system that aligns artificial intelligence-driven policy generation with the imperative of sustainable industrial operation and risk mitigation in the insurance industry. The PAIL model, through its intricate components and feedback mechanisms, presents a progressive approach to integrating technological innovation with environmental consciousness and risk assessment accuracy, heralding a new era of intelligent, sustainable industrial and insurance practices.

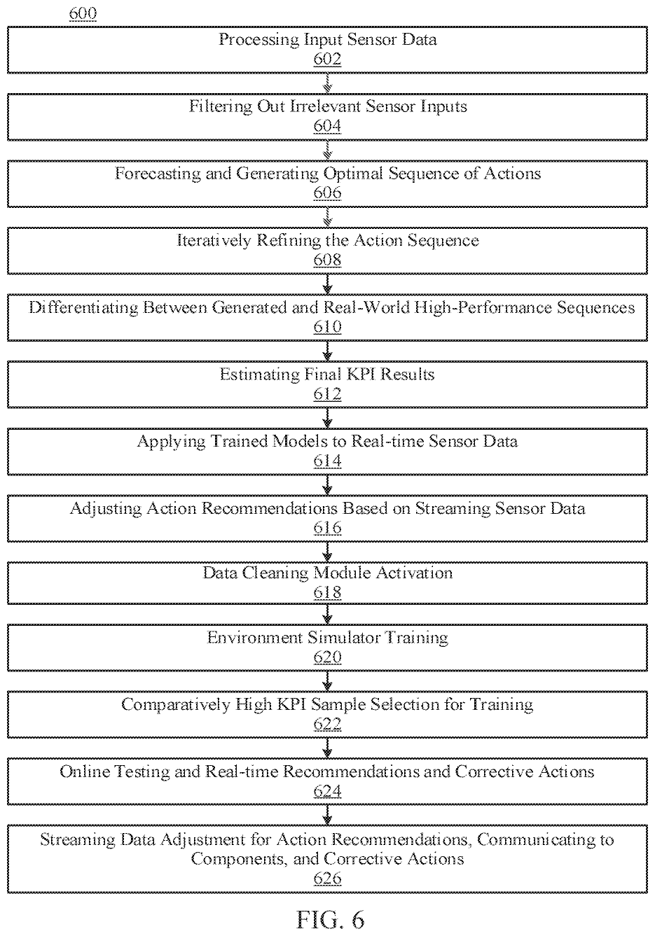

Referring now to FIG. 6, a block/flow diagram showing a method 600 for dynamically optimizing Key Performance Indicators (KPI) for one or more systems and processes by integrating offline training and online testing devices using Performance-based Adversarial Imitation Learning (PAIL) for dynamic optimization of industrial processes and personalized risk assessment, is illustratively depicted in accordance with embodiments of the present invention.

In various embodiments, in block 602, input sensor data from one or more sensors monitoring an industrial process or asset can be processed in accordance with aspects of the present invention. This step involves collecting and analyzing data from various sensors deployed across the industrial setting. These sensors may measure a range of variables including temperature, pressure, humidity, speed, driver actions and other environmental or operational parameters relevant to the industrial process. The processing step can prepare the sensor data for subsequent analysis, which can include normalization, segmentation, and filtering to ensure the data is in a suitable format for the predictive models to utilize effectively.

In block 604, following the initial processing, sensor inputs which are determined to be irrelevant based on their correlation to the final KPI can be filtered out. This involves analyzing the historical impact of each sensor's readings on the KPIs and excluding data from sensors that do not significantly affect the outcome. The correlation analysis helps in identifying which variables are most predictive of the KPIs, thereby streamlining the dataset to enhance the accuracy and efficiency of the forecasting model.

In block 606, a policy generator network with a transformer-based architecture can be utilized to forecast and generate an optimal sequence of actions based on the results of the processed input sensor data. This step involves leveraging the transformer's ability to handle sequential data, applying its self-attention mechanism to discern patterns and dependencies in the historical sensor data. The policy generator predicts future actions that could optimize the KPIs, iteratively refining these predictions through simulation to form an action sequence that is believed to achieve the best possible outcome.

In block 608, the forecasting and generation of the optimal sequence of actions can include a process of iteratively refining the action sequence through simulation, leveraging historical data. This iterative refinement involves simulating the effects of proposed actions on the industrial process, using historical data to predict the outcomes. Adjustments are made to the sequence based on the simulation results, with the goal of converging on an action plan that maximizes KPI performance.

In block 610, a discriminator network utilizing a neural network architecture can be employed to differentiate between generated action sequences and real-world high-performance sequences. This step assesses the quality and realism of the generated action sequences by comparing them to a dataset of sequences known to have resulted in high KPI performance. The discriminator's feedback is used to further refine the policy generator's output, encouraging it to produce action sequences that more closely mimic those that have historically led to success.

In block 612, final KPI results can be estimated based on the generated action sequences using a performance prediction network. This network, also based on a transformer architecture, computes the reward of each action within the sequence by estimating its impact on the final KPI. This involves evaluating how each proposed action, and the sequence as a whole, is likely to influence the KPIs, allowing for the optimization of the action plan towards achieving the best possible performance.

In block 614, the trained models can be applied to real-time sensor data to recommend actions for ongoing industrial processes. This step translates the insights gained from historical data analysis and simulation into actionable recommendations for live operations. The system uses current sensor readings to dynamically generate advice on the optimal actions to take at any given moment, aiming to continuously optimize the KPIs.

In block 616, actions recommendations for ongoing industrial processes can include adjusting action recommendations based on streaming sensor data to achieve real-time optimization of KPIs. This involves a feedback loop where the system continuously monitors the effect of implemented actions on the KPIs and adjusts its future recommendations accordingly. The goal is to maintain or improve KPI performance by dynamically responding to changes in the industrial process or external conditions, ensuring the operational strategy remains aligned with the optimization objectives.

In block 618, a data cleaning module can be activated and can preprocess the sensor data by removing noise and irrelevant information. This module evaluates the statistical relevance of each sensor's data to the KPI and discards data from sensors with minimal or no impact, ensuring that only pertinent information is utilized in further processing.

In block 620, an environment simulator can be trained using variational autoencoder (VAE) techniques to simulate future states of the industrial process based on current actions and states. This simulator aids the policy generator by providing a predictive model of the process's behavior under various conditions, allowing for more informed decision-making regarding action sequences.

In block 622, the system can select comparatively high KPI samples from historical data to train the policy generator and discriminator networks. This step focuses the learning process on successful examples, enabling the networks to learn the characteristics and action sequences that lead to high-performance outcomes.

In block 624, trained models can be applied to real-time sensor data to recommend actions for ongoing industrial processes. This involves using the trained policy generator and performance predictor to evaluate current process conditions and propose actions designed to optimize KPIs in real time.

In block 626, action recommendations and/or automatic corrective actions can be adjusted and/or performed based on streaming sensor data to achieve real-time optimization of KPIs. This dynamic adjustment process allows the system to respond to changes in the industrial process environment or operational conditions, ensuring that the action sequences remain optimized for current conditions, in accordance with aspects of the present invention. In an embodiment, a status of low-risk due to action sequences can be communicated directly to components or customers, e.g., by computer (e.g., email, a telephone call, snail mail, etc.), although other communicating methods can be employed. In this way, the corrective action or beneficial action can be performed by the components, customer, entity, which can take advantage of their low-risk status designation.

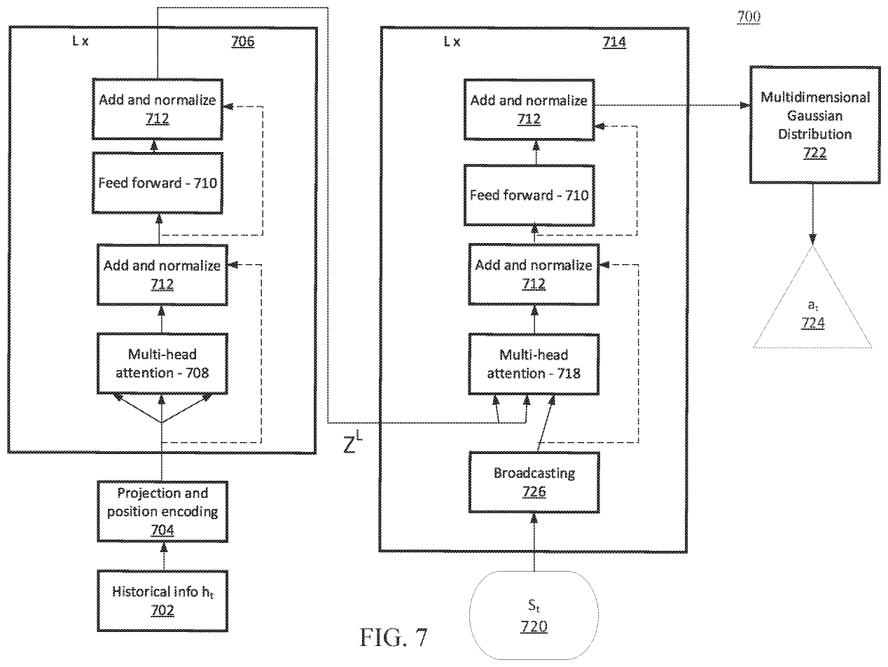

Referring now to FIG. 7, a diagram showing a framework of a system and method 700 for action sequence simulation and generation using an Adversarial Imitation Learning Engine (AILE) for dynamic personalized driver action risk assessment is illustratively depicted in accordance with embodiments of the present invention.

In various embodiments, it is noted that real-world driver's actions are significantly influenced by previous actions and the states of the car and environments. As an example, in highway driving, prior actions, such as brake or throttle activations, as well as a car's state (e.g., the speed of the car, type of car, age of car), can significantly shape probabilities of subsequent actions on the same car (e.g., push throttle again to speed up or brake to speed down). In light of such temporal dependencies, an adapted Transformer architecture can be employed to generate action sequences with discerning temporal correlations in a trajectory. A multi-head self-attention architecture enables simultaneous processing of multiple trajectories. It is particularly beneficial for the capture of subtle and long-range inter-dependencies in the trajectories. A trajectory is the dataset of both the action sequence and a time series of a sensor reading. This attribute is needed for precise modeling along a temporal dimension where context and historical trends are paramount. Furthermore, given the task of forecasting new action sequences with historic trajectories, the self-attention mechanism exhibits superiority to dynamically adjusting focused segments of an input.

For each trajectory ti, let us partition the trajectory by a fixed window length T. A window sequence Xᵢ={x₁, x₂, . . . , x_{T}} is obtained. Here, each xₜ in Xᵢ includes two factors: a concatenated state sₜ and an action vector at in the window. Note that, the length of the input sequence Xₜ, representing historical information 702, varies for each time step t. To address this challenge, a sliding window methodology is employed to select the preceding 1 elements for action prediction, here l is a hyper-parameter. Thus, the historical information at time step t can be written as hₜ=xₜ₋₁, . . . , xₜ₋₁.

In block 704, a projection of the input Hᵢ=h₁, . . . , h_{T} into spaces of query Q, key K, and value V via projection matrices W_{q}, Wₖ, and Wᵥ in R^{d×d} is performed. The correlation across time steps within the sequence is computed as follows:

$${Z}_{i} = soft max ( \frac{{QK}^{T}}{\sqrt{{d}_{k}}} ) V = soft max ( \frac{( {H}_{i} {W}_{q} ) {( {H}_{i} {W}_{k} )}^{T}}{\sqrt{{d}_{k}}} ) ( {H}_{i} {W}_{v} )$$

In the foundational Transformer model, positional information is incorporated using sinusoidal position encoding (block 704). In the Transformer framework, these representations traverse L layers in block 706, and are subjected to both multi-head self-attention (block 708) and position-wise feed-forward operations (block 710). Formally, for each layer l=1, . . . , L:

$${Z}^{l} = LayerNorm ( {Z}^{l - 1} + MultiHead ( {Q}^{l - 1} , {K}^{l - 1} , {V}^{l - 1} ) )$$

$${Z}^{l} = LayerNorm ( {Z}^{l} + FFN ( {Z}^{l} ) ) .$$

LayerNorm is a normalization function that normalizes the sum of the arguments (blocks 712) as each layer (l) is traversed. The multi-head attention mechanism 708 partitions Z into h segments and integrates the output of these individual heads in layers 714 by:

$$MultiHead ( Q , K , V ) = Concat ( {head}_{1} , … , {head}_{h} ) {W}_{O} ,$$

where head j=Attention (Qj, Kj, Vj) and WO is a learned projection matrix.

After the Transformer encoder is deployed on temporal input data, an output representation HL captures intricate temporal inter-dependencies across various timestamps. Next, a decoder can be designed to elevate the model's proficiency in processing complex sequence data for action prediction. The decoder architecture incorporates a multi-head cross-attention module 718. It is operated by dynamically focusing on correlated segments of the historical trajectory in relation to the current state sₜ. The structural and functional dynamics of the cross-attention module 718 are similar to the previous self-attention module 708. The decoder output representation matrix Z′ᵢ of trajectory τᵢ is computed as follows:

$${Z}_{i}^{′} = softmax ( \frac{{Q}_{i} {K}_{i}^{T}}{\sqrt{{d}_{k}}} ) {V}_{i} = softmax ( \frac{( {S}_{i} {W}_{q}^{′} ) {( {Z}_{i}^{L} {W}_{k}^{′} )}^{T}}{\sqrt{{d}_{k}}} ) ( {Z}_{i}^{L} {W}_{v}^{′} )$$

In the above equation, Zᵢ^{L} denotes encoding historical information of trajectory τᵢ, Zᵢ^{L}=z₁, . . . , z_{T}, and Sᵢ denotes a broadcasting matrix 726 of a current state 720 (sₜ). The output of multi-head cross attention module 718 is also obtained by concatenating the outputs from all heads and projecting them through a linear layer.

In the unique scenario of driver's action sequence generation task, a continuous action space encompasses multiple inter-related action types. Unlike traditional solutions to employ the sigmoid function for categorical prediction, an objective is to allocate a distinct value to each type of action. Hence, an action vector 722 is created while simultaneously expanding exploration space for these actions. An assumption can be made that the actions adhere to a multivariate distribution. Based on this distribution learned by an output layer, a recommended action 724 at current timestamp (at), as shown in the following equation can be determined:

$${π}_{θ} ( a \vert h , s ) = p ( a ; μ , \sum )$$

where μ and Σ represent the mean vector and covariance matrix of the actions, respectively. They are the output of the dense layer with the input Z′, in accordance with aspects of the present invention.

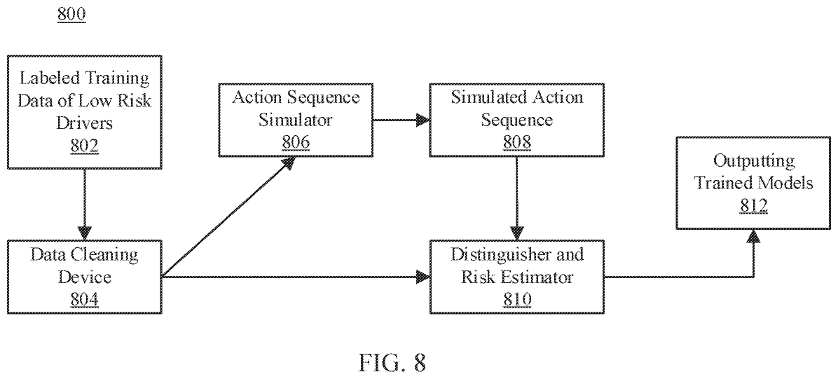

Referring now to FIG. 8, a diagram showing a system and method 800 for model training using an Adversarial Imitation Learning Engine (AILE) for dynamic personalized driver action risk assessment is illustratively depicted in accordance with embodiments of the present invention.

In various embodiments, during model training, the AILE can train an action distinguisher and risk estimator from labeled driver data. There can be several components utilized at this stage, including, for example, a data cleaning device/module 804 to clean the sensor data, an action sequence simulator 806 to generate the action sequences, and distinguisher module to distinguish the generated sequences from the real ones, in accordance with aspects of the present invention.

In various embodiments, the problem of auto insurance risk factor estimation can be defined as follows: Input: Vehicle sensor datasets, including two parts: (1) a comparatively small dataset D1 with the labels of “low risk driver”; and (2) a comparatively large dataset D2 without any labels. For the dataset D1, “trajectories” can be used to refer to the historical actions of the drivers, and the sensor recordings (in format of time series). Trajectory={action sequences, sensor time series}. Output: (1) For each driver in D2, the labels of “low/high risk drivers” can be added; (2) the suggested insurance premium; and (3) explanation information to support the decisions in (1) and (2), in accordance with aspects of the present invention.

In block 802, labeled training data of low-risk drivers, the AILE system begins the risk estimation process. This data can be utilized as a foundational benchmark of what constitutes low-risk driving behavior. It can include, for example, comprehensive sensor and action sequence information from vehicles operated by drivers who have been classified based on historical records and patterns as having low risk profiles. This data may include metrics such as steady acceleration patterns, smooth cornering, adherence to speed limits, and consistent following distances, which have been correlated with safer driving outcomes and fewer insurance claims. Historic driving data can also be employed, which can include driving infractions or other pertinent information.

In block 804, a data cleaning device undertakes the critical task of ensuring the integrity and relevance of the training data. This involves sophisticated algorithms designed to filter out extraneous noise, correct errors, and normalize the data for consistent analysis. The data cleaning process is extensive, involving outlier detection, error rectification, smoothing algorithms for time-series data, and the harmonization of data formats across various sensor inputs. This cleansing paves the way for more accurate modeling and simulation of driver behavior, as it ensures the data used for training reflects true driving conditions without distortions that could lead to skewed risk assessments. A data preprocessing module can be employed to cleanse the dataset and eliminate sensor readings affected by noisy conditions. Within the vehicle system, numerous sensors continuously monitor the environment and produce readings. However, not all of these readings are indicative of associated risks of the drivers.

In various embodiments, for each vehicle sensor, the statistics of the time series can be retrieved, including, e.g., the average, standard deviation, max value and min value. Then, a table as shown in Table 3 can be built and the Pearson correlation of each feature can be computed to determine a final label of the driver. Here, a numerical value can be employed to give the driver a score. If a driver has never filed any claim or receive any tickets, the score can be 100, if the driver has a speeding ticket in the past five years, the score can be 90, etc. (The higher the score, the better the driver's performance). If the absolute value of correlation is close to 0, it indicates that the value of the sensor is not related to driver's performance score, and AILE can filter out such a sensor.

Exemplary Table 3 lists the statistics of sensor s1 to driver's score. Assume in the training dataset that there are 90 driver's samples with labels. For each driver, AILE retrieves the sensor data (time series) of s1 and compute the statistics as shown in Table 3 below. Then, AILE computes the Pearson coefficient of each statistic to the performance score. In Table 3, the coefficient between average value and performance score is 0.76, and the coefficient between median and performance score is 0.78. This indicates that the value of sensor s1 has a strong relationship with the driver's performance score. Hence, sensor s1 will not be filtered out by the data cleaning module in this example.

TABLE 3

| | | | | | | Driver |
|---|---|---|---|---|---|---|
| | Average | STD | Max | Min | Median | score |
| Sample 1 | 66 | 21 | 90 | 60 | 60 | 70 |
| Sample 2 | 64 | 12 | 80 | 55 | 56 | 68 |
| Sample 3 | 53 | 17 | 70 | 45 | 47 | 57 |
| . . . | . . . | . . . | . . . | . . . | . . . | . . . |
| Sample 90 | 80 | 16 | 96 | 67 | 75 | 75 |

In block 806, the action sequence simulator, having received cleansed and curated data, actively simulates possible driver actions. The simulator applies complex predictive models, potentially including stochastic models, to estimate future actions based on historical patterns. This simulation considers numerous scenarios, factoring in variables like road conditions, traffic patterns, and typical driver responses to create a comprehensive set of possible future driving sequences. These sequences are employed for training the system to anticipate and evaluate the potential risk associated with different driving behaviors.

In block 808, the simulated action sequence generated by the action sequence simulator provides the system with hypothetical yet plausible sequences of driver actions, which are used for further analysis and model training. These sequences are synthesized to represent a wide array of driving behaviors and conditions, serving as a virtual testing ground for the AILE system to evaluate and learn from.

In block 810, a distinguisher and risk estimator can be a dual-component system where the distinguisher can critically evaluate the simulated action sequences against known benchmarks of low-risk behavior to ensure authenticity and accuracy. Simultaneously, the risk estimator component can appraise each action within the sequence, ascribing risk scores based on a complex matrix of factors such as the abruptness of the action, the situational context, and historical correlation with incidents.

In various embodiments, in block 810, there can be three main components, including a state simulator configured for estimating the influence of each action, including estimating the vehicle state after a driver conducts an action; a driver discriminator configured for distinguishing the generated action sequences (e.g., simulated driver) and the real driver with high performance score (e.g., low risks); and a driver's performance estimator configured to take input of the drivers' trajectories and output the estimated performance scores, in accordance with aspects of the present invention.

In various embodiments, the driver's risk estimator in block 810 can perform precise predictions on the state evolutions following conducted actions in the trajectory (e.g., if the driver presses the throttle, the car's speed will increase). In pursuit of modeling this predictive framework, a Variational AutoEncoder (VAE) can be utilized, and the VAE is a deep generative model that captures potential future states following the actions. For a given state sₜ and a designated action aₜ, the encoder of the VAE maps the state-action pairing into a latent space as:

$${q}_{ϕ} ( z \vert {s}_{t} , {a}_{t} ) = 𝒩 ( z ; μ ( {s}_{t} , {a}_{t} ) , {σ}^{2} ( {s}_{t} , {a}_{t} ) )$$

where μ and σ are mean and variance of z.

A core objective during training is to approximate the true posterior distribution. It aims to minimize the discrepancy between predicted future states and observed outcomes, formulated as pθ(st+1|st, at, z). The VAE integrates a regularization term to prevent overfitting and ensure a smoother latent space:

$$ℒ = {𝔼}_{{q}_{ϕ}} [ log {p}_{θ} ( {s}_{t + 1} \vert {s}_{t} , {a}_{t} , z ) ] - β KL ( {q}_{ϕ} ( z \vert {s}_{t} , {a}_{t} ) \vert \vert 𝒩 ( 0 , I ) )$$

where β is a hyper-parameter to balance the two terms in the loss function. This VAE network (e.g., state simulator based on actions) can be trained prior to the training of AILE's modules. Once it achieves a predefined performance criterion, its parameters can be fixed to ensure consistent interactions during subsequent learning stages. With this simulation framework, the consequences of certain actions can be accurately predicted, thereby informing the decision making process and paving the way for crafting optimal operational sequences, in accordance with aspects of the present invention.

In various embodiments, in block 810, AILE can utilize a discriminator to distinguish between the drivers' trajectories with high performance and those ones generated by the model. By trying to distinguish the real trajectory, the discriminator catches the key features of the high performance trajectories. AILE integrates the historical information together with current state to generate the recommended actions. The historical vector can be obtained by applying average pooling on the original vectors, denoted as h. AILE can leverage the lω-parameterized multiple-layer perception (MLP) D_{lω}(h,s,a). This function estimates the likelihood of a given history-state-action tuple (h,s,a) originating from a trajectory of high performance.

In various embodiments, the discriminator can address a binary classification task, and both the policy generator and the discriminator can engage in a Min-Max game predicated on the cross-entropy loss.

The objective of discriminator can be:

$${max}_{ω} {𝔼}_{{π}_{E}} [ log {D}_{ω} ( h , s , a ) ] + {𝔼}_{{π}_{θ}} [ log ( 1 - {D}_{ω} ( h , s , a ) ) ]$$

The objective of action sequence generator can be:

$${L}_{IL} = {min}_{θ} {𝔼}_{{π}_{θ}} [ log ( 1 - {D}_{ω} ( h , s , a ) ) ]$$

In real world applications, the trajectories with high performance (e.g., low risk of accidents and claims) are more rare than the ones with middle or low performance (e.g., high risks). The limited training data poses a challenge to the imitation learning framework, and to address this issue, a performance-oriented training guidance mechanism for the action sequence generator can be utilized. This mechanism can maximize the cumulative performance value of each state-action pair, enhancing the overall performance of generated trajectories. A key feature is to derive a reward signal for each discrete timestamp and interpret the discriminator output as an inverse measure of the performance. Formally, the reward signal for a history-state action tuple (h,s,a) is quantified as R(h,s,a)=−log (Da(h,s,a). This formulation establishes a relationship such that when the discriminator assigns a value close to 0, indicating significant deviation from existing trajectories of high performance, the corresponding reward is a large negative value. This penalizes the action sequence generator for low-performance actions, guiding it towards high-performance recommendations. Subsequently, performance estimator can be utilized to accurately and efficiently assess the value of each history-state action tuple.

In various embodiments, block 810 can further include a performance estimator. In real industrial systems and vehicle systems, quantifying the immediate performance credit of each single action in a long trajectory is non-trivial. In most scenarios, the historical trajectories only have a final performance value as the overall performance. This challenge of attributing performance credit to distinct actions has led us to exploit Temporal Difference (TD) learning for a deep Q network. The network can estimate the utility of any given state-action pair. Specifically, a refined self-attention network F can be utilized to capture historical inter-dependence across time and assess the performance credits for all the actions in the trajectory.

The network architecture can be similar to the encoder of action sequence generator, and it can be pre-trained by minimizing the square loss to existing trajectories with high performance. For example, for trajectory t with length of N, the overall performance for state s and action a can be calculated as:

$$V ( τ ) = F ( {s}_{1} , {a}_{1} , … , {s}_{N} , {a}_{N} )$$

Hence the immediate reward at timestamp t can be obtained by a discriminator as follows:

$${r}_{t} = - {𝔼}_{( h , s , a ) ∼ {π}_{θ}} log ( {D}_{ω} ( {h}_{t} , {s}_{t} , {a}_{t} ) )$$

In order to evaluate the performance credit of a specific state-action pair, we initialize a Q-network, denoted by Q (s, a|θ^{Q}) with arbitrary parameters. Meanwhile, we define the target value network as Q\hat{ }|prime (s, a|θΛQ\hat{ }|prime), The Temporal Difference (TD) error, crucial for updating the Q network, is calculated as follows:

$${δ}_{t} = {r}_{t} + γ {Q}^{′} ( {s}_{t + 1} , {a}_{t + 1} \vert {θ}^{{Q}^{′}} ) - Q ( {s}_{t} , {a}_{t} \vert {θ}^{Q} )$$

$${δ}_{T} = {r}_{T} - Q ( {s}_{T} , {a}_{T} \vert {θ}^{Q} ) = V ( τ ) - Q ( {s}_{T} , {a}_{T} \vert {θ}^{Q} )$$

where γ represents the discount factor to capture the present performance score and Q′(sₜ₊₁, aₜ₊₁|θ^{Q′}) denotes the performance score of next state action pair estimated by the target network.

To encourage exploration and prevent premature convergence to sub-optimal policies, AILE introduces Gaussian noise to the deterministic action output of the policy network and uses them as the input to the target network Q′. In this way, AILE facilitates strategic exploration of the action space to optimize Q-value of subsequent state-action pairs. A goal of TD learning is to minimize the temporal difference (TD) error. Accordingly, the loss function for TD learning module is formulated as:

$$ℒ ( {θ}^{Q} ) = {𝔼}_{( s , a ) ∼ {π}_{θ}} [ {δ}_{t}^{2} ] ,$$

where δₜ represents the TD error at time t. The parameter updating steps of both Q and Q\hat{ }{prime} networks are synchronized with the imitation learning module. This learning process involves multiple iterations of updates in a single epoch, delineated as follows:

$${θ}^{Q} ← {θ}^{Q} - η {∇}_{θ Q} ℒ ( {θ}^{Q} ) ,$$

$${θ}^{{Q}^{′}} ← {ϵθ}^{Q} + ( 1 - ϵ ) {θ}^{{Q}^{′}} ,$$

where ϵ denotes the learning rate for gradient descent in the module, and Q′ network is softly updated by copying the parameters from the Q network. ϵ is set as a small constant (e.g., 0.01) to ensure gradual updates. In essence, a goal is to optimize a neural architecture and infer the performance score of different state-action pairs. The guide is on the maximization of the Q-value for recommended actions, as shown in the following equation:

$${L}_{v a l u e} = - {E}_{a ∼ {π}_{θ} ( \cdot \vert s )} [ Q ( s , a ) ]$$

Thus, the loss function for the policy generator is:

L=λL_{IL}+(1−λ)Lᵥₐₗᵤₑ+β(t)H(π) where λ is a hyper-parameter to moderate the relative importance of the two objectives, and H (Tt) is the entropy of learned policy. In AILE framework, the entropy regularization term is dynamically adjusted using a decay function for the time-dependent coefficient β(t). AILE uses an exponential decay function β(t)=β₀e^{-kt}, where β₀ is the initial value, k is the decay rate, and t represents the epoch. This exponential decay allows for aggressive exploration in the initial phases of training and progressively shifts the focus towards exploitation by reducing the influence of the entropy over time. As a result, the action sequence generator derives its learning signal by back-propagating from both the discriminator and the performance estimator. This dual influence ensures that the generator not only emulates the trajectories of high performance score but also ensures each action is optimized for highest performance improvement.

In block 812, trained models can be output from the executed training process, where the AILE system can encapsulate its learned knowledge into sophisticated models ready for application to any of a plurality of situations. These models, now fine-tuned, are adept at processing raw, real-time sensor data from vehicles to accurately estimate the risk profile of drivers. The system employs these models to score and classify new drivers, identifying those with behaviors that closely match the low-risk patterns the system was trained on.

In various embodiments, the AILE system can dynamically employ advanced algorithms and learning techniques in real-time to ensure that the training data accurately informs the system's understanding of low-risk driving behavior. The detailed nature of the data, combined with the comprehensive approach to cleaning, simulation, and generation of action sequences, ensures that the AILE system can effectively differentiate between low and high-risk drivers with high accuracy. The output trained models encapsulate the distilled wisdom of the AILE system, manifesting as a suite of advanced algorithms that can seamlessly analyze new, unlabeled sensor data to discern driving risk in real-time. These models can be utilized in real-time to assign real-time risk assessments, capable of recognizing and flagging emerging patterns that mirror the high-risk or low-risk driving behaviors they have been trained to detect, in accordance with aspects of the present invention.

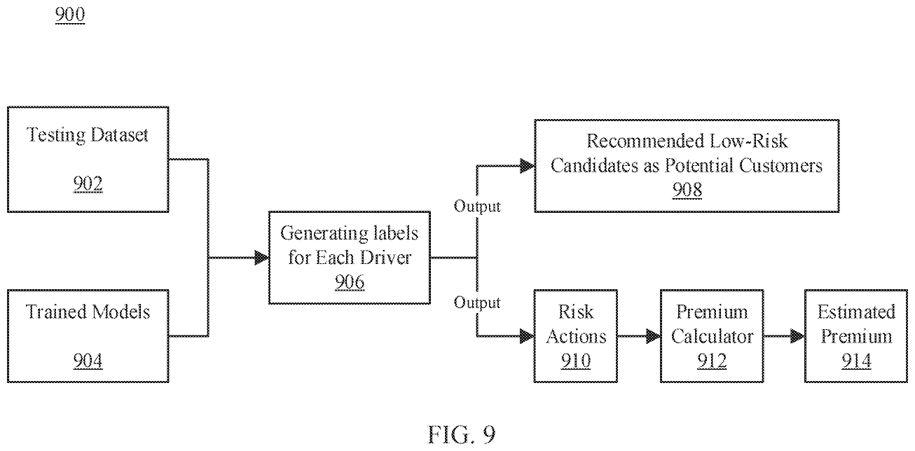

Referring now to FIG. 9, a diagram showing a high-level view of a system and method 900 for real-time end user/customer searching and monitoring using an Adversarial Imitation Learning Engine (AILE) for dynamic personalized driver action risk assessment is illustratively depicted in accordance with embodiments of the present invention.

In various embodiments, in block 902, a testing dataset can be used as input, and can be a comprehensive array of sensor data collected from vehicles during operation. This dataset is rich in detail, providing a temporal sequence of events and actions that accurately represent real-world driving conditions and behaviors. The data can include, but is not limited to, speed variations, braking intensity, steering angles, and turn signal usage, which are useful for the subsequent risk assessment process. In block 904, the system can leverage trained models that have been rigorously developed and validated using substantial historical datasets. These models can incorporate advanced algorithms designed to detect, analyze, and interpret complex driving patterns, making them robust tools for evaluating real-time vehicle sensor data. The models can be fine-tuned to identify nuances in driving behaviors that contribute to risk profiles, in accordance with aspects of the present invention.

In block 906, a sophisticated labeling process can be initiated, where the trained models are applied to the testing dataset to generate predictive risk assessments for each driver. This labeling process involves classifying drivers based on the potential risk associated with their driving behaviors, utilizing a multi-faceted analysis to discern between low and high-risk profiles with high accuracy. With the discriminator and performance estimator trained, now AILE can process the unlabeled drivers' trajectories. AILE can generate the performance scores and the estimate their risks. The high-performance drivers with low risks are selected as potential customer candidates. AILE can use the distinguisher to select out the trajectories of the good performances, and then use the performance estimator to estimate the scores. Finally, the performance of the driver Pi and estimated as the mean of all his/her trajectories as follows:

$$Performance ( {P}_{i} ) = {mean}_{j i n Tra ( Pi )} ( Performance ( {tra}_{i j} ) )$$

In block 908, drivers deemed as low-risk by the system can be earmarked as recommended candidates for auto insurance providers. The criteria for selection can be derived from the analysis conducted by the AI models, which can consider, for example, a driver's adherence to safe driving practices and historical patterns that suggest a lower probability of filing insurance claims.

In block 910, a detailed analysis of the driving actions of an individual or group of individuals (e.g., a company fleet) can be conducted to evaluate risk. Each action, such as sudden acceleration or hard braking, can be analyzed within the context of its occurrence to determine its contribution to overall driving risk. The actions can be scored based on their risk levels, with higher scores reflecting higher potential risk and vice versa, in accordance with aspects of the present invention.

In block 912, a sophisticated premium estimator and calculator can be employed, and can integrate the risk scores associated with individual driving actions using actuarial science combined with machine learning insights to accurately calculate personalized insurance premiums and/or generate personalized insurance leads for potential new customers for an insurance provider. It can consider various risk factors and personalize the premium rates and insurance leads to match the assessed risk level for each driver.

In various embodiments, the insurance premium can be estimated/calculated based on driver's performance and risk factors. For an example driver, we will partition his/her driving history into M different time windows (e.g., 1 year). Assume that there are totally M different time windows. The final premium of driver Pi in term T is computed as follows:

$$Premium ( {P}_{i} ) = {u}_{i} + Claim\_cost ( {P}_{i} ) * {\sum }_{m = 1}^{M} Performance ( {P}_{i} , {T}_{m} ) * Damage ( {e}_{m} )$$

where uᵢ is a base cost, Claim_cost (Pᵢ) is the cost of selected cost by the driver, e.g., liability, comprehensive cost of the vehicle, etc. Damage(eₘ) is the estimated damage that a risky driving action can cause (e.g., the incorrect parking can cause a 50% damage to the vehicle, and the action of speeding may cause 100% damage to the vehicle value). Therefore, the total loss can be calculated as a product of the claim_cost and the sum of all the risk factors, in accordance with aspects of the present invention.

In block 914, the estimated premium for each driver (including potential new customers identified as leads) can be computed and output by the system. This calculation can include the assessment of driving behavior, the identification of risk-related actions, and their corresponding impact on insurance risk. The output provides auto insurance companies with an actionable insight into the potential risk each driver presents, enabling them to offer premiums that are commensurate with the assessed risk level, in accordance with aspects of the present invention.

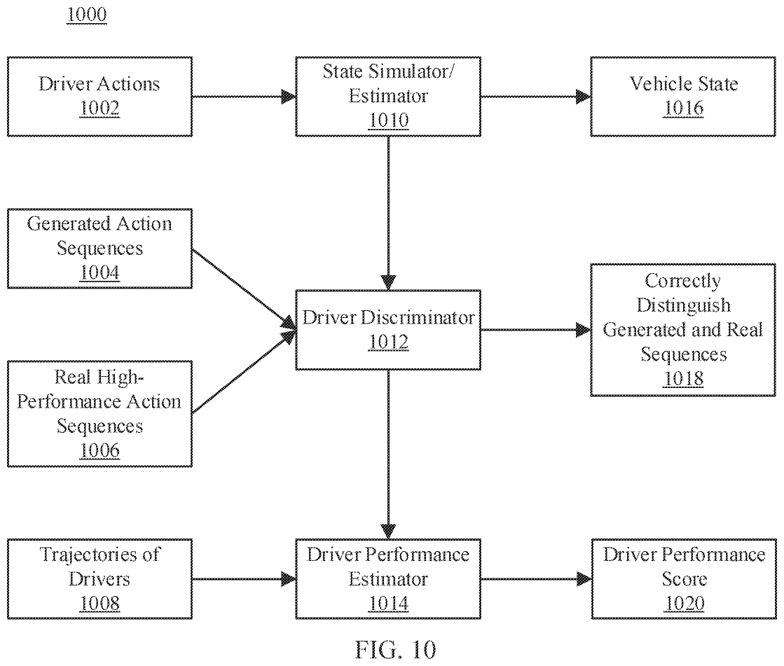

Referring now to FIG. 10, a diagram showing a framework of a system and method 1000 for dynamically optimizing personalized driver action risk assessment based on a distinguisher and risk estimator using an Adversarial Imitation Learning Engine (AILE) is illustratively depicted in accordance with embodiments of the present invention.

In various embodiments, in block 1002, driver actions can be collected, capturing a comprehensive array of real-time and historical driver behaviors from a network of onboard vehicle sensors. This data may include detailed sequences and patterns of braking, acceleration, turning, and lane-keeping, providing a robust dataset crucial for the nuanced assessment of driving patterns and tendencies. In some embodiments, camera data, satellite date and other types of date can be included.

In block 1004, the system, utilizing advanced algorithms, can generate action sequences that reflect possible driving behaviors based on the observed data. These synthesized sequences are constructed to mirror real-world driving scenarios, thereby enabling the predictive analysis of driver responses under various conditions.

In block 1006, real high-performance action sequences are compiled from a curated dataset of historical driving data. These sequences represent the benchmark of safe driving behaviors and are utilized to calibrate the system's understanding of risk, serving as a standard against which generated sequences can be measured. In block 1008, the complete trajectories of drivers can be documented, encapsulating the sequence of driver actions, vehicle responses, and contextual environmental factors. These trajectories are integral to the system's ability to map out the dynamic nature of driving behavior over time.

In block 1010, a sophisticated state simulator/estimator component can utilize the input of driver actions and calculate the corresponding vehicle state changes. This simulation can include predictive modeling of potential outcomes, factoring in the complexities of various driving situations and their implications for vehicle safety and performance. In block 1012, the driver discriminator can critically evaluate the action sequences (in real-time or for later processing), using advanced pattern recognition and machine learning techniques to differentiate between high-risk and low-risk driving patterns. This component's discerning analysis can be pivotal in refining the system's risk estimation algorithms, in accordance with aspects of the present invention.

In block 1014, the driver performance and risk estimator can assess each driver action, calculating individual risk scores and correlating them with specific driving behaviors. It can integrate these scores to formulate a composite risk profile for the driver, effectively enabling a detailed risk assessment. In block 1016, contextual vehicle state data can be analyzed, providing important information about the vehicle's operating conditions at the time of each driver action. This data adds a layer of depth to the risk assessment, allowing the system to adjust risk scores based on the vehicle's response to driver inputs in real-time.

In block 1018, the system can be utilized to accurately distinguish between generated and real sequences. This capability is important in ensuring the artificial intelligence (AI) model's accuracy in identifying characteristic patterns of low-risk driving behaviors. In block 1020, the system can calculate a comprehensive driver performance score. This score is a metric which can be derived from the risk estimator's analysis, reflecting the cumulative risk associated with an individual's driving behavior. This score can serve as a key determinant in the formulation of insurance premiums and determinations of particular individuals and/or entities to target as insurance leads, with lower scores suggesting safer driving habits and potentially qualifying drivers for reduced insurance costs or being offered insurance policies, in accordance with aspects of the present invention.

It should be understood that the systems and models described herein can be updated continuously or intermittently. The amount of computational resources and accuracy of the model can influence updating schedules.

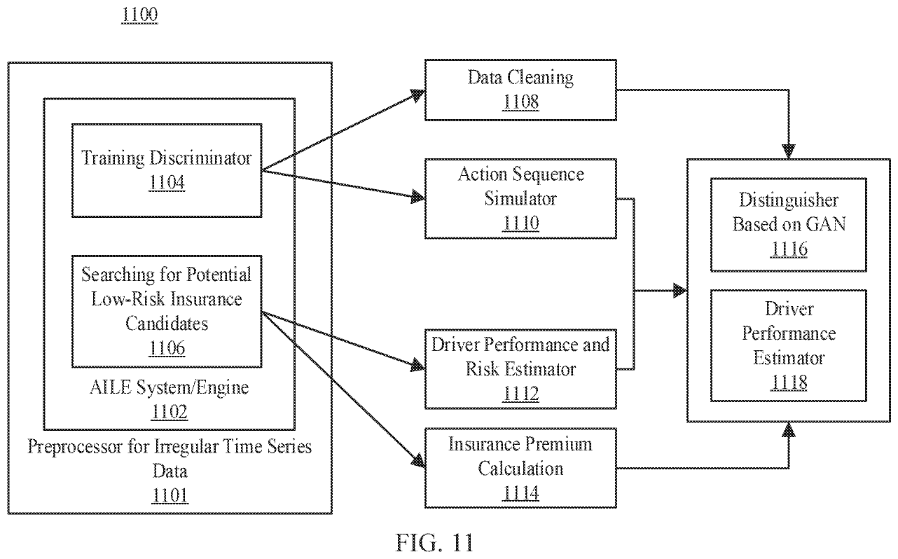

Referring now to FIG. 11, a diagram showing a system and method for optimizing Key Performance Indicators (KPI) for one or more systems and processes by integrating offline training and online testing devices using an Adversarial Imitation Learning Engine (AILE) for dynamic personalized driver action risk assessment is illustratively depicted in accordance with embodiments of the present invention.

In various embodiments, the system and method 1100 can utilize an Adversarial Imitation Learning Engine (AILE) for driver action risk estimation, targeting the identification of low-risk insurance candidates by evaluating vehicle sensor data, etc., in accordance with aspects of the present invention. In various embodiments, the AILE System/Engine 1102 serves as the central processing unit within the framework, orchestrating the flow of data and information through the various components of the system to assess driver behavior and risk. At the initial stage, the Preprocessor for Irregular Time Series Data 1101 can prepare and normalize the sensor data received from the vehicles or other sources, ensuring it is in an appropriate format for further analysis. The sensor data can be received over a wired or wireless network by a receiving device, which can include a computer system or telecommunications receiver or any other data collection method or device.

The Training Discriminator 1104 can be utilized for distinguishing between high-risk and low-risk driving patterns. It processes historical and real-time data to learn the distinguishing characteristics of driving behavior that correlate with lower insurance claims. In conjunction with the discriminator, the Action Sequence Simulator 1110 can utilize the processed sensor data to generate potential future action sequences for the driver. This simulation can be utilized to predict the likelihood of a driver engaging in high or low-risk behaviors, in accordance with aspects of the present invention.

The Searching for Potential Low-Risk Insurance Candidates in block 1106 can include applying the learned models to new, unlabeled sensor data to identify drivers who exhibit characteristics similar to the low-risk profiles learned during the training phase. In block 1108, a Data Cleaning module can refine the sensor data further, removing anomalies and ensuring that only the comparatively most relevant and accurate data is used for risk estimation. Once potential action sequences are simulated, in block 1116, the Distinguisher Based on a Generative Adversarial Network (GAN) can utilize the GAN to refine the ability of the system to differentiate between high and low-risk driving behaviors, effectively learning to mimic the action sequences of known low-risk drivers, in accordance with aspects of the present invention.

The Driver Performance and Risk Estimator 1112 can analyze and process the output from the GAN and provide a risk score for each simulated driver action sequence. This estimator pinpoints specific actions within the sequence that contribute to the overall risk assessment. A final step can include the Insurance Premium Calculation 1114, which utilizes the risk scores and the identified driver behaviors to estimate appropriate auto insurance premiums for potential low-risk drivers. In accordance with various embodiments, the system and method 1100 provide an innovative approach to the auto insurance industry's challenge of identifying and selecting potential low-risk drivers for risk mitigation in offering insurance policies and adjusting existing policies based on driver and/or vehicle performance. By leveraging the granular data collected from vehicle sensors, the AILE system enables insurers to make informed decisions backed by AI-driven risk assessments, and can be adjusted in real-time based on real-time sensor measurements deployed in a vehicle. This solves traditional problems of reliance on historical records and personal data by providing a dynamic, real-time analysis that reflects the current risk profile of drivers, in accordance with aspects of the present invention.

It should be understood that while the present embodiments illustrative describe applications in auto insurance, the present embodiments can be applied to any type of form of insurance or any other application where risk assessment and behavior prediction are employed.

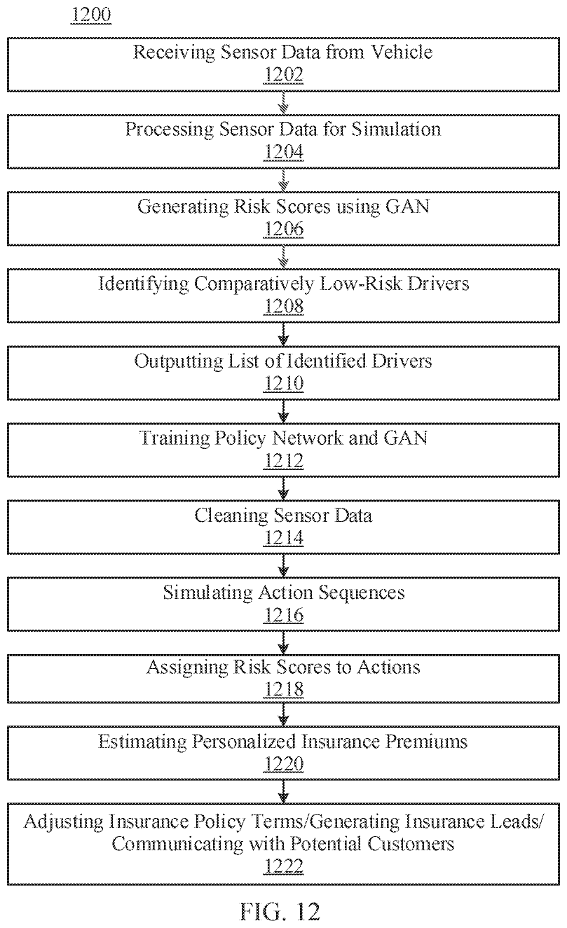

Referring now to FIG. 12, a block/flow diagram showing a method 1200 for optimizing Key Performance Indicators (KPI) for one or more systems and processes by integrating offline training and online testing devices using an Adversarial Imitation Learning Engine (AILE) for dynamic personalized driver action risk assessment is illustratively depicted in accordance with embodiments of the present invention.

In various embodiments, in block 1202, sensor data from a vehicle can be received by a computing system that is configured to receive and process sensor data from a vehicle. A sensor receiver can include any memory storage device or system or any real-time receiving system for receiving information updates from a plurality of sensors for components of a system being monitored. including but not limited to a network This data comprises information about the state of the vehicle, such as speed, acceleration, and location, as well as actions of the driver, including braking intensity, steering angle, turn signal use, etc.

The received sensor data can be processed by the computing system in block 1204 to simulate a driver action sequence. This processing involves a transformer-based policy network that encodes temporal dependencies within the sensor data. The network uses multi-head self-attention mechanisms to process sequential sensor inputs, accommodating the dynamic nature of driving behavior. In block 1206, a risk score for the simulated driver action sequence can be generated by the computing system using a Generative Adversarial Network (GAN). The GAN is composed of a generator that creates action sequences and a discriminator that discerns generated action sequences from real low-risk action sequences, promoting the generation of realistic driving behavior patterns.

In block 1208, the computing system can identify potential low-risk drivers based on the generated risk scores. These determinations are made through an analytical engine that assesses the likelihood of actions leading to incidents, thereby flagging drivers who exhibit safer driving patterns. In block 1210, a list of potential low-risk drivers is output by the computing system, serving as candidates for auto insurance premium estimation. This list is a compilation of drivers whose simulated actions, as per the risk assessment model, align with the characteristics of low-risk driving.

In block 1212, the transformer-based policy network and GAN can be trained through several steps. Initially, the policy network is modeled to simulate realistic driver action sequences based on labeled datasets of known low-risk drivers. The generator within the GAN is trained to produce diverse and realistic driver action sequences, and the discriminator is optimized to distinguish real from synthetic sequences. This training includes a feedback loop where the discriminator's assessments refine the generator's outputs for better simulation accuracy.

In block 1214, sensor data received by the system can be cleaned by computing statistical measures such as average, standard deviation, and extremes, and applying a Pearson correlation coefficient. This step filters out data that does not correlate with driver performance scores, ensuring the focus is on relevant sensor inputs for risk assessment. In block 1216, the processed sensor data can be used to simulate a driver action sequences, employing an adapted transformer architecture that discerns temporal correlations. This enables the capture of nuanced and extended patterns within the driver's action sequences, reflecting the intricate nature of driving behaviors.

In block 1218, a performance prediction neural network can assign risk scores to individual driver actions within the sequence. This network analyzes actions in the context of their temporal sequence, providing a detailed understanding of how each action contributes to the overall risk profile. In block 1220, insurance premiums for potential low-risk drivers can be estimated by aggregating risk scores across multiple driver action sequences. The premiums are adjusted according to quantified risk factors associated with the sequences, enabling personalized premium calculations.

In block 1222, the system can utilize the generated risk scores to adjust insurance policy terms directly for potential low-risk drivers and/or generate insurance leads, in accordance with aspects of the present invention. This can include modifications to deductibles, coverage limits, and premium rates, and/or offering new customers insurance packages based on insurance leads generated by the system, and reflecting the quantified risk levels, in accordance with aspects of the present invention. For example, an insurer could employ the systems and methods of the present invention to determine potential low-risk customers and reach out to these customers with a discounted rate or other incentive to subscribe for insurance. A status of low-risk due to action sequences can be communicated directly to potential customers, e.g., a communication device. The communication device can include, e.g., a computer (e.g., email) a telecommunications device (e.g., cell phone), snail mail, etc.

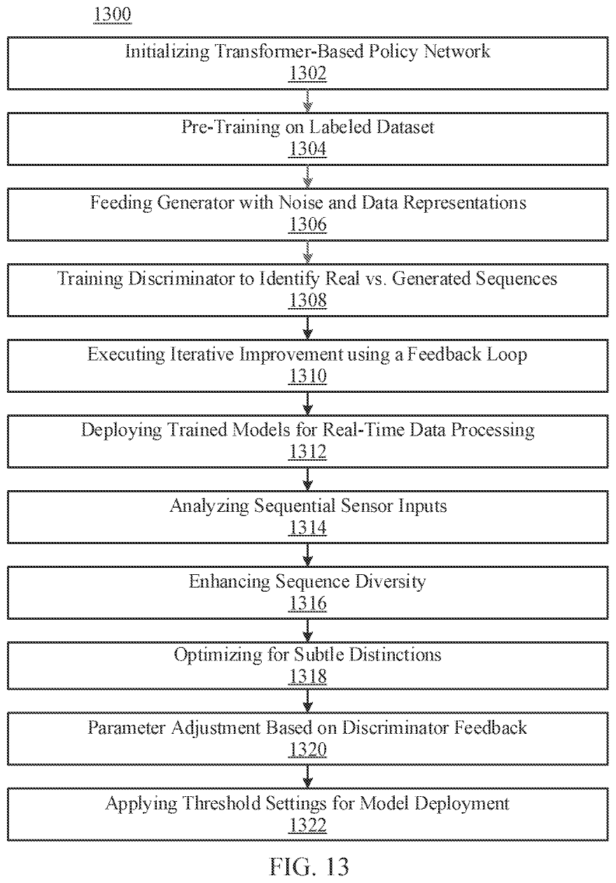

Referring now to FIG. 13, a block/flow diagram showing a method 1300 for training Artificial Intelligence (AI) models for dynamic, real-time personalized driver action risk assessment using an Adversarial Imitation Learning Engine (AILE) is illustratively depicted in accordance with embodiments of the present invention.

In block 1302, a transformer-based policy network can be initialized by a computing system. The network models driver action sequences by encoding temporal dependencies found within sensor data. It processes sequential sensor inputs using a multi-head self-attention mechanism, drawing from a pre-trained model on labeled data of known low-risk drivers to simulate realistic driving actions. In block 1304, the policy network can undergo pre-training on a dataset comprising sensor data from verified low-risk drivers. This phase involves adjusting network weights to accurately replicate the driving patterns that correspond with low-risk behaviors.

In block 1306, a computing system can train the generator within the GAN by feeding it with noise vectors and latent representations of sensor data. This process employs an adapted transformer architecture designed to produce diverse and realistic driver action sequences that resemble the behavior of low-risk drivers. In block 1308, the discriminator within the GAN is trained concurrently to distinguish real driver action sequences from the synthetic ones produced by the generator. The computing system employs a CNN architecture within the discriminator optimized for recognizing subtle distinctions, enhancing its ability to identify authentic low-risk driving patterns.

In block 1310, a feedback loop can be employed where the computing system uses assessments from the discriminator to fine-tune the generator's parameters. This iterative improvement process is configured to refine the generated action sequences, aiming to make them indistinguishable from actual low-risk driver actions. In block 1312, upon reaching a predetermined accuracy threshold for distinguishing between real and synthetic action sequences, the trained transformer-based policy network and GAN are deployed by the computing system. They are utilized for processing incoming unlabeled sensor data, generating real-time risk scores, and identifying potential low-risk drivers.

In block 1314, the transformer-based policy network can analyze sequential sensor inputs with attention to the temporal context of each input, ensuring that the generated action sequences reflect the temporal patterns observed in low-risk driving. In block 1316, the generator can utilize mechanisms to enhance the diversity and realism of the generated action sequences. This includes altering the noise vectors and adjusting the model to encourage variation in the generated sequences.

In block 1318, the computing system can optimize the discriminator to identify subtle distinctions in action sequences, which is essential for distinguishing between low-risk and potential high-risk driving behaviors. In block 1320, parameters within the policy network and GAN are adjusted based on feedback from the discriminator. This fine-tuning ensures the models are learning effectively and improving their predictive accuracy over time.

In block 1322, the computing system can set an accuracy threshold that the models must reach before being deployed for real-time data processing. This threshold ensures the reliability of the models in live environments. FIG. 13 outlines the training pipeline from initializing and pre-training the models to deploying them for real-time assessment, and including the detailed mechanisms and methodologies employed to enhance model accuracy, speed, and overall performance, and ensure the accuracy of risk score generation, in accordance with aspects of the present invention.

Referring now to FIG. 14, a diagram showing a high-level view of a system for optimizing Key Performance Indicators (KPI) for one or more systems and processes by integrating offline training and online testing devices using an Adversarial Imitation Learning Engine (AILE) for dynamic personalized driver action risk assessment is illustratively depicted in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment,” as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for training a transformer-based policy network and Generative Adversarial Network (GAN), comprising:
initializing a transformer-based policy network to model action sequences by encoding temporal dependencies within sensor data, where the transformer-based policy network utilizes multi-head self-attention mechanisms to process sequential sensor inputs, the multi-head self-attention mechanisms being pre-trained on a labeled dataset having sensor data from known low-risk action sequences;
training a generator within the GAN to produce generated action sequences by feeding noise and latent representations of sensor data, where the generator employs an adapted transformer architecture to enhance diversity and realism of the generated action sequences, which mimic behavior of low-risk action sequences;
concurrently training a discriminator within the GAN to differentiate between action sequences derived from the labeled dataset and synthetic action sequences produced by the generator;
employing a feedback loop where assessments by the discriminator are used to adjust parameters for an iterative improvement process configured to produce sequences indistinguishable from real low-risk action sequences; and
deploying the trained transformer-based policy network and GAN to process incoming unlabeled sensor data for real-time generation of risk scores and identification of low-risk action sequences upon reaching a predetermined threshold for accuracy in distinguishing between real and synthetic action sequences.

2. The method of claim 1, wherein training includes monitoring vehicles to collect the sensor data where the action sequences include driver actions.

3. The method of claim 2, wherein the action sequences include historical driver actions.

4. The method of claim 1, further comprising:
cleaning the sensor data by computing statistical measures on the sensor data; and
filtering the sensor data to remove unrelated data by employing a Pearson correlation coefficient computation.

5. The method of claim 1, wherein the sensor data includes action sequences and temporal correlations are discerned using an adapted Transformer architecture to capture subtle and long-range dependencies within the action sequences.

6. The method of claim 1, where the discriminator utilizes a convolutional neural network (CNN) architecture optimized to identify subtle distinctions between real and generated action sequences.

7. The method of claim 1, further comprising utilizing a performance prediction neural network to assign risk scores to individual actions and identify low-risk action sequences, where the neural network is based on a Transformer architecture that performs analysis of actions in a context of their temporal sequence.

8. The method of claim 7, further comprising estimating an insurance premium for drivers in accordance with the risk scores.

9. The method of claim 7, further comprising employing the risk scores to directly adjust insurance policy terms for potential low-risk drivers.

10. A system for training a transformer-based policy network and Generative Adversarial Network (GAN), comprising:
a hardware processor; and
a memory that stores a computer program which, when executed by the hardware processor, causes the hardware processor to:
initialize a transformer-based policy network to model action sequences by encoding temporal dependencies within sensor data, where the transformer-based policy network utilizes multi-head self-attention mechanisms to process sequential sensor inputs, the multi-head self-attention mechanisms being pre-trained on a labeled dataset having sensor data from known low-risk action sequences;
train a generator within the GAN to produce generated action sequences by feeding noise and latent representations of sensor data, where the generator employs an adapted transformer architecture to enhance diversity and realism of the generated action sequences, which mimic behavior of low-risk action sequences;
concurrently train a discriminator within the GAN to differentiate between action sequences derived from the labeled dataset and synthetic action sequences produced by the generator;
utilize a feedback loop where assessments by the discriminator are used to adjust parameters for an iterative improvement process configured to produce sequences indistinguishable from real low-risk action sequences; and
deploying the trained transformer-based policy network and GAN to process incoming unlabeled sensor data for real-time generation of risk scores and identification of low-risk action sequences upon reaching a predetermined threshold for accuracy in distinguishing between real and synthetic action sequences.

11. The system of claim 10, wherein the hardware processor monitors vehicles to collect the sensor data where the action sequences include driver actions.

12. The system of claim 11, wherein the action sequences include historical driver actions.

13. The system of claim 10, wherein the computer program causes the hardware processor to clean the sensor data by computing statistical measures on the sensor data; and filter the sensor data to remove unrelated data by employing a Pearson correlation coefficient computation.

14. The system of claim 10, wherein the sensor data includes action sequences and temporal correlations are discerned using an adapted Transformer architecture to capture subtle and long-range dependencies within the action sequences.

15. The system of claim 10, where the discriminator utilizes a convolutional neural network (CNN) architecture optimized to identify subtle distinctions between real and generated action sequences.

16. The system of claim 10, wherein the computer program causes the hardware processor to utilize a performance prediction neural network to assign risk scores to individual actions and identify low-risk action sequences, where the neural network is based on a Transformer architecture that performs analysis of actions in a context of their temporal sequence.

17. The system of claim 16, further comprising estimating an insurance premium for drivers in accordance with the risk scores.

18. The system of claim 16, further comprising employing the risk scores to directly adjust insurance policy terms for potential low-risk drivers.

19. The system of claim 10, wherein the transformer-based policy network employs a self-attention mechanism that enables simultaneous processing of multiple trajectories for modelling complex driver behaviors over time.

20. A computer-readable medium storing instructions that, when executed by a processor, perform a method for training a transformer-based policy network and Generative Adversarial Network (GAN), comprising:
initializing a transformer-based policy network to model action sequences by encoding temporal dependencies within sensor data, where the transformer-based policy network utilizes multi-head self-attention mechanisms to process sequential sensor inputs, the multi-head self-attention mechanisms being pre-trained on a labeled dataset having sensor data from known low-risk action sequences;
training a generator within the GAN to produce generated action sequences by feeding noise and latent representations of sensor data, where the generator employs an adapted transformer architecture to enhance diversity and realism of the generated action sequences, which mimic behavior of low-risk action sequences;
concurrently training a discriminator within the GAN to differentiate between action sequences derived from the labeled dataset and synthetic action sequences produced by the generator;
employing a feedback loop where assessments by the discriminator are used to adjust parameters for an iterative improvement process configured to produce sequences indistinguishable from real low-risk action sequences; and
deploying the trained transformer-based policy network and GAN to process incoming unlabeled sensor data for real-time generation of risk scores and identification of low-risk action sequences upon reaching a predetermined threshold for accuracy in distinguishing between real and synthetic action sequences.
